SEQUENCE LISTING (1 sequence)

SEQ ID NO: 1 - protein, 8 aa, synthetic construct
AQTGTGKT

(19) United States
(12) Patent Application Publication
(10) Pub. No.: US 2025/0213645 A1
(43) Pub. Date: Jul. 3, 2025

(54) PHARMACEUTICAL COMPOSITION FOR MODULATING IMMUNE RESPONSE

(71) Applicant: L-BASE CO., LTD., Seoul (KR)

(72) Inventor: Do Yong JEON, Seoul (KR)

(21) Appl. No.: 18/692,883

(22) PCT Filed: Sep. 16, 2022
(86) PCT No.: PCT/KR2022/013901

§ 371 Date: Mar. 14, 2025

(30) Foreign Application Priority Data
Sep. 17, 2021 (KR) 10-2021-0125238
Sep. 15, 2022 (KR) 10-2022-0116655

(51) Int. Cl. A61K 38/08; A61K 39/395; A61P 35/00

(52) U.S. Cl. CPC A61K 38/08; A61K 39/3955; A61P 35/00

(57) ABSTRACT

The present invention relates to a pharmaceutical composition for enhancing immunity, and was arrived at by discovering that oligopeptide AQTGTGKT and an analog thereof have the effect of approximately modulating immune activity in the body, such as by suppressing excessive immune response while enhancing immune activity for defending the body from cancer or infectious diseases and the like. In particular, the compound according to the present invention can significantly improve the sensitivity of a subject to an immune anticancer drug, activate immune cells in a tumor microenvironment through the regulation of cytokines, chemokines, etc., and inhibit excessive autophagy activity to control physiological balance, and thus using the compound in combination with immune anti-cancer drugs can maximize the anticancer effect thereof. Therefore, the compound according to the present invention can optimize the body's defense function and enhance the effect of immune anticancer drugs and the like through the control of immune response, and thus is expected to be utilized in the treatment of various immune diseases and cancer.

### TECHNICAL FIELD

The present invention relates to a pharmaceutical composition for immune enhancement and a pharmaceutical composition for enhancing the anticancer effect of a cancer immunotherapeutic agent.

This application claims priority to and the benefit of Korean Patent Application No. 10-2021-0125238, filed on Sep. 17, 2021, and Korean Patent Application No. 10-2022-0116655, filed on Sep. 15, 2022, the disclosures of which are incorporated herein by reference in their entirety.

### BACKGROUND ART

Although the effectiveness of cancer treatment is currently improving due to the development of early cancer diagnosis methods and the continuous development of new anticancer therapies, cancer is still an important disease that ranks first or second among the causes of death in Korea. Most of the anticancer drugs that are currently used are chemotherapy-based and have varying pharmacological effects depending on the type of cancer, and they exhibit various side effects due to toxicity, which is pointed out as a problem in cancer treatment.

As an example of an anticancer drug, antibodies that target specific tumor antigens of tumor cells without affecting normal tissue are being developed. However, antibodies have problems such as concerns about immune responses and low efficiency of tissue penetration. On the other hand, unlike antibodies, peptides are less likely to cause immune responses and easily penetrate into tissue due to a small molecular weight, and since a peptide-based anticancer drug targeting a tumor antigen can selectively act on a tumor, it is expected that there will be virtually no side effects, such as damage to normal cells.

However, despite these advantages, such peptide-based anticancer drugs are only used for a very limited range of cancer types, and particularly have a problem of being decomposed within a short time immediately after administration to humans, making it difficult to exhibit their effects.

As a method for solving the above problems, by modifying a peptide through amidation, esterification, acylation, acetylation, PEGylation, cyclization, or alkylation, it can improve yield, increase in vivo activity, increase the affinity of the peptide for a receptor, and delay protein degradation. However, even with the above modification, it is not guaranteed that the desired effect will be enhanced, and there is a risk that the peptide may lose its original in vivo activity or cause unexpected side effects. Accordingly, research on peptide modification to minimize such negative effects, while simultaneously maximizing the desired effects, is actively progressing.

Meanwhile, combination therapy for cancer treatment has an advantage of attacking cancer cells via multiple means, so it is widely used in cancer treatment. Combination therapy can enhance the efficacy of anticancer drugs and reduce the amount of the administered anticancer drugs, thereby minimizing the toxicity and side effects of the anticancer drugs, and it is also useful when resistance to anticancer drugs appears. However, despite the fact that a number of effective combination therapies have been identified over the past several decades, the number of people dying from cancer each year continues to rise, making it important to develop effective combination therapy.

Recently, the development of anticancer treatment methods which induce immune cells to more effectively recognize and attack cancer cells by regulating a patient's immune system rather than directly attacking cancer cells with a chemotherapy drug is underway. Cancer immunotherapeutic agents that have been developed as part of this refer to anticancer drugs that restore or strengthen the tumor recognition or destruction ability of the immune system for cancer cells to overcome an immunosuppression or immune escape mechanism acquired by cancer cells through genetic changes. However, when a cancer immunotherapy drug is used alone, problems such as a patient not responding to treatment or developing stronger resistance may occur. Accordingly, research is being actively conducted to enhance sensitivity to cancer immunotherapy drug monotherapy, but an alternative method or combination therapy for enhancing sensitivity to cancer immunotherapy drug monotherapy has not yet been discovered.

### DISCLOSURE

### Technical Problem

The present invention was devised to solve the above problems and completed after confirming that a compound according to the present invention, such as the oligopeptide AQTGTGKT, and an analog thereof have an effect of appropriately regulating immune activity in the body, such as enhancing immune activity for body defense while suppressing excessive immune responses, and particularly, an effect of enhancing the sensitivity of a subject to a cancer immunotherapy drug and thus can be used as a composition for co-administration of cancer immunotherapy drugs.

Therefore, the present invention is directed to providing a pharmaceutical composition for immune enhancement, which includes an oligopeptide consisting of the amino acid sequence of SEQ ID NO: 1 or an analog thereof as an active ingredient.

The present invention is also directed to providing a pharmaceutical composition for preventing or treating cancer, which includes an oligopeptide consisting of the amino acid sequence of SEQ ID NO: 1 or an analog thereof as an active ingredient, and a kit including the same.

The present invention is also directed to providing a pharmaceutical composition for enhancing the anticancer effect of a cancer immunotherapeutic agent, which includes an oligopeptide consisting of the amino acid sequence of SEQ ID NO: 1 or an analog thereof as an active ingredient, and a kit for co-administration of a cancer immunotherapy drug, which includes the same.

However, technical problems to be solved in the present invention are not limited to the above-described problems, and other problems which are not described herein will be fully understood by those of ordinary skill in the art from the following descriptions.

### Technical Solution

The present invention provides a pharmaceutical composition for immune enhancement, which includes an oligopeptide consisting of the amino acid sequence of SEQ ID NO: 1 or an analog thereof as an active ingredient.

The present invention also provides a method for immune enhancement, which includes administering an oligopeptide consisting of the amino acid sequence of SEQ ID NO: 1 or an analog thereof; or a composition including the same to a subject in need thereof.

The present invention also provides a use of an oligopeptide consisting of the amino acid sequence of SEQ ID NO: 1 or an analog thereof; or a composition including the same for immune enhancement.

The present invention also provides a use of an oligopeptide consisting of the amino acid sequence of SEQ ID NO: 1 or an analog thereof; or a composition including the same for preparing an immune enhancer.

The present invention also provides a pharmaceutical composition for preventing or treating cancer, which includes an oligopeptide consisting of the amino acid sequence of SEQ ID NO: 1 or an analog thereof; and a cancer immunotherapy drug as active ingredients.

The present invention also provides a kit for preventing or treating cancer, which includes the oligopeptide or an analog thereof; or a composition including the same.

The present invention also provides a method of preventing or treating cancer, which includes administering an oligopeptide consisting of the amino acid sequence of SEQ ID NO: 1 or an analog thereof; and a cancer immunotherapy drug (or a composition including the same) to a subject in need thereof.

The present invention also provides a use of an oligopeptide consisting of the amino acid sequence of SEQ ID NO: 1 or an analog thereof; and a cancer immunotherapy drug (or a composition including the same) for preventing or treating cancer.

The present invention also provides a use of an oligopeptide consisting of the amino acid sequence of SEQ ID NO: 1 or an analog thereof; and a cancer immunotherapy drug (or a composition including the same) for preparing a drug for treating cancer.

The present invention also provides a pharmaceutical composition for enhancing the anticancer effect of a cancer immunotherapeutic agent, which includes an oligopeptide consisting of the amino acid sequence of SEQ ID NO: 1 or an analog thereof as an active ingredient.

The present invention also provides a use of an oligopeptide consisting of the amino acid sequence of SEQ ID NO: 1 or an analog thereof for enhancing the anticancer effect of a cancer immunotherapy drug.

The present invention also provides a method of enhancing the anticancer effect of a cancer immunotherapy drug, which includes administering an oligopeptide consisting of the amino acid sequence of SEQ ID NO: 1 or an analog thereof, to a subject in need thereof.

The present invention also provides a use of an oligopeptide consisting of the amino acid sequence of SEQ ID NO: 1 or an analog thereof for preparing an anticancer effect enhancer (or adjuvant) of a cancer immunotherapy drug.

The present invention also provides a pharmaceutical composition for co-administering a cancer immunotherapy drug, which includes an oligopeptide consisting of the amino acid sequence of SEQ ID NO: 1 or an analog thereof as an active ingredient.

The present invention also provides a combined use of an oligopeptide consisting of the amino acid sequence of SEQ ID NO: 1 or an analog thereof with a cancer immunotherapy drug.

In one embodiment of the present invention, the analog is a compound represented by General Formula X-AQTGTGKT, wherein X may be a compound represented by Chemical Formula 1 below, or a pharmaceutically acceptable salt thereof, but the present invention is not limited thereto:

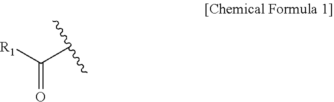

In Chemical Formula 1,
R₁ may be hydrogen, a substituted or unsubstituted phenyl group, a substituted or unsubstituted naphthyl group, or a substituted or unsubstituted C₁ to C₅ alkyl group.

In another embodiment of the present invention, R₁ may be a compound represented by any one of Chemical Formulas 2 to 4 below, but the present invention is not limited thereto:

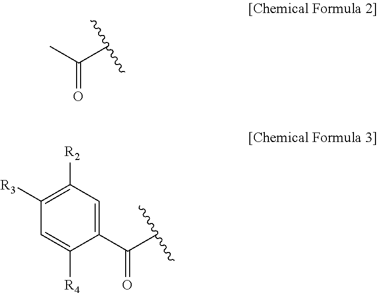

(In Chemical Formula 3,
R₂ and R₄ are each independently hydrogen, a C₁ to C₅ alkyl group, or a phenyl group; and
R₃ is hydrogen, a C₁ to C₅ alkyl group, a phenyl group, or a C₁ to C₃ alkoxy group.)

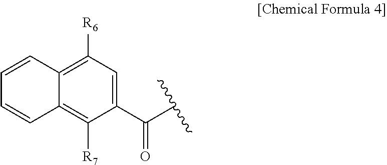

(In Chemical Formula 4,
R₆ and R₇ are each independently hydrogen or a C₁ to C₃ alkyl group.)

In still another embodiment of the present invention, the oligopeptide or an analog thereof may satisfy one or more characteristics selected from the group consisting of the following, but the present invention is not limited thereto:
(a) increasing an immune response against one or more selected from the group consisting of tumor antigens, bacterial infections, and viral infections;
(b) suppressing immune hypersensitivity;
(c) regulating the level or activity of one or more selected from the group consisting of CXCL10, IFNγ, TNFα, CCL5, CXCL9, CXCL11, IL-1α, IL-1β, IL-6, and IL-12; and
(d) increasing the level or activity of one or more selected from the group consisting of cytotoxic T cells and M2 macrophages.

In yet another embodiment of the present invention, the cancer immunotherapy drug may be one or more selected from the group consisting of an immune checkpoint inhibitor, a costimulatory molecule agonist, a cytokine therapeutic, a CAR-T cell therapeutic, and an autologous CD8+T immune cell therapeutic, but the present invention is not limited thereto.

In yet another embodiment of the present invention, the immune checkpoint inhibitor may be one or more selected from the group consisting of a PD-L1 inhibitor, a PD-1 inhibitor, a CTLA-4 inhibitor, a LAG3 inhibitor, a TIM3 inhibitor, a 4-1BB inhibitor, a LAG-3 inhibitor, a B7-H4 inhibitor, an HVEM inhibitor, a TIM4 inhibitor, a GAL9 inhibitor, a VISTA inhibitor, a KIR inhibitor, a TIGIT inhibitor, and a BTLA inhibitor, but the present invention is not limited thereto.

In yet another embodiment of the present invention, the immune checkpoint inhibitor may be one or more selected from the group consisting of atezolizumab, avelumab, dostarlimab, durvalumab, ipilimumab, nivolumab, and pembrolizumab, but the present invention is not limited thereto.

In yet another embodiment of the present invention, the costimulatory molecule agonist may be one or more selected from the group consisting of a 4-1BB inhibitor, and an OX40 inhibitor, but the present invention is not limited thereto.

In yet another embodiment of the present invention, the composition may be in the form of a mixture in which the oligopeptide or an analog thereof; and the cancer immunotherapy drug are mixed, but the present invention is not limited thereto.

In yet another embodiment of the present invention, the composition may be designed such that each of the oligopeptide or analog thereof and the cancer immunotherapy drug is prepared and simultaneously, separately, or sequentially administered, but the present invention is not limited thereto.

In yet another embodiment of the present invention, the composition may be administered to a subject at a dose such that the cancer immunotherapy drug is administered at a concentration of 0.1 to 50 mg/kg, but the present invention is not limited thereto.

In yet another embodiment of the present invention, the composition may be administered to a subject at a dose such that the oligopeptide or an analog thereof is administered at a concentration of 0.01 to 500 mg/kg, but the present invention is not limited thereto.

In yet another embodiment of the present invention, the cancer immunotherapy drug:the oligopeptide or an analog thereof may be included at a weight ratio of 1:0.1 to 10, but the present invention is not limited thereto.

In yet another embodiment of the present invention, the composition may satisfy one or more characteristics selected from the group consisting of the following, but the present invention is not limited thereto:
(a) an increase in the level or activity of tumor-suppressive immune cells;
(b) a reduction in the level of immunosuppressive immune cells; and
(c) the regulation of the level or activity of one or more selected from the group consisting of CXCL10, IFNγ, TNFα, CCL5, CXCL9, CXCL11, IL-1α, IL-1β, IL-6, and IL-12.

In yet another embodiment of the present invention, the cancer may be one or more selected from the group consisting of squamous cell carcinoma, lung cancer, adenocarcinoma of the lung, peritoneal cancer, skin cancer, skin or intraocular melanoma, rectal cancer, perianal cancer, esophageal cancer, small intestine cancer, endocrine cancer, parathyroid cancer, adrenal cancer, soft tissue sarcoma, urethral cancer, blood cancer, liver cancer, gastrointestinal cancer, pancreatic cancer, glioblastoma, cervical cancer, ovarian cancer, bladder cancer, hepatocellular carcinoma, breast cancer, colon cancer, colorectal cancer, endometrial cancer, uterine cancer, salivary gland cancer, kidney cancer, prostate cancer, vulvar cancer, thyroid cancer, head and neck cancer, and brain cancer, but the present invention is not limited thereto.

In yet another embodiment of the present invention, the cancer may be one or more selected from the group consisting of microsatellite instability-high (MSS-high) mutation, microsatellite instability-low (MSI-low) mutation, and microsatellite stability (MSS) mutation, but the present invention is not limited thereto.

In yet another embodiment of the present invention, the composition may include two or more cancer immunotherapy drugs, but the present invention is not limited thereto.

In yet another embodiment of the present invention, the oligopeptide of the present invention or an analog thereof; or a composition including the same may be simultaneously, separately, or sequentially administered with a cancer immunotherapy drug, but the present invention is not limited thereto.

### Advantageous Effects

The present invention relates to a pharmaceutical composition for immune enhancement, and has been completed by confirming that the oligopeptide AQTGTGKT and an analog thereof appropriately regulates immune activity in the body, such as enhancing immune activity for body defense and suppressing excessive immune responses, in the case of cancer, infectious diseases, etc. Particularly, the compound according to the present invention can significantly enhance the sensitivity of a subject to a cancer immunotherapy drug, and adjust a physiological balance by activating immune cells in a tumor microenvironment through the regulation of cytokines or chemokines and suppressing excessive autophagy activity, thereby maximizing its anticancer effect in combination with the cancer immunotherapy drug. Therefore, since the compound according to the present invention can optimize the defense function of the body and enhance the effect of the cancer immunotherapy drug through the regulation of immune responses, it is expected to be used in the treatment of various immune diseases and cancer.

### DESCRIPTION OF DRAWINGS

FIGS. 1 to 7 are diagrams showing UPLC-MS (upper) and ¹H NMR (lower) results for identifying compounds according to the present invention and confirm the structure of the compounds, in which FIG. 1 shows the results of measuring 4-PhPh-AQTGTGKT, FIG. 2 shows the results of measuring Ac-AQTGTGKT, FIG. 3 shows the results of measuring 3-PhPh-AQTGTGKT, FIG. 4 shows the results of measuring 4-MeOPh-AQTGTGKT, FIG. 5 shows the results of measuring 2-PhPh-AQTGTGKT, FIG. 6 shows the results of measuring Ph-AQTGTGKT, and FIG. 7 shows the results of measuring Naphthyl-AQTGTGKT.

FIGS. 8A and 8B show the results of comparing anticancer efficacy according to the single or combined administration of a compound (AQTGTGKT) according to the present invention and a cancer immunotherapy drug in cancer animal models produced using CAGE-expressing cancer cells (FIG. 8A, tumor growth curve; FIG. 8B, tumor weight measurement results).

FIGS. 9A to 9C show the results of comparing anticancer efficacy according to the single or combined administration of a compound (3-PhPh-AQTGTGKT) according to the present invention and a cancer immunotherapy drug in cancer animal models produced using CAGE-expressing cancer cells (FIG. 9A, tumor growth curve; and FIG. 9B, tumor weight measurement results).

FIGS. 10A to 10C show the results of comparing anticancer efficacy according to the single or combined administration of a compound (Naphthyl-AQTGTGKT) according to the present invention and a cancer immunotherapy drug in cancer animal models produced using CAGE-expressing cancer cells (FIG. 10A, tumor growth curve; and FIG. 10B, tumor weight measurement results).

FIG. 11A shows the results of H&E staining for confirming the degree of cancer cell death in tumor tissue after single or combined administration of a compound (3-PhPh-AQTGTGKT) according to the present invention and a cancer immunotherapy drug in cancer animal models produced using CAGE-expressing cancer cells.

FIGS. 11B and 11C show the results of H&E staining for confirming the frequencies of T cells (FIG. 11B) and macrophages (FIG. 11C) in tumor tissue after single or combined administration of a compound (3-PhPh-AQTGTGKT) according to the present invention and a cancer immunotherapy drug in cancer animal models produced using CAGE-expressing cancer cells.

FIG. 12 shows the results of measuring an mRNA expression level of CXCL10 in tumor tissue after single or combined administration of a compound (3-PhPh-AQTGTGKT) according to the present invention and a cancer immunotherapy drug in cancer animal models produced using CT26 cancer cells in which CAGE expression was induced.

FIGS. 13A and 13B show the results of comparing anticancer efficacy after single or combined administration of a compound (3-PhPh-AQTGTGKT) according to the present invention and a cancer immunotherapy drug in cancer animal models produced using CT26 cancer cells in which CAGE expression was induced (FIG. 13A, tumor growth curve; FIG. 13B, tumor weight measurement results)

FIGS. 14 and 15 show the results of comparing an overall survival (OS) period after single or combined administration of a compound (3-PhPh-AQTGTGKT) according to the present invention and a cancer immunotherapy drug in cancer animal models produced using CT26 cancer cells in which CAGE expression was induced.

FIG. 16 shows the results of comparing an overall survival (OS) period after single or combined administration of a compound (3-PhPh-AQTGTGKT) according to the present invention and two types of cancer immunotherapy drugs in cancer animal models produced using CT26 cancer cells in which CAGE expression was induced.

### MODES OF THE INVENTION

The present invention relates to a pharmaceutical composition for immune enhancement, and has been completed by confirming that an oligopeptide AQTGTGKT and an analog thereof have an effect of increasing immune activity in the body by enhancing immune activity for body defense. Particularly, it was confirmed that, when used in combination with a cancer immunotherapy drug, the compound according to the present invention can not only maximize the anticancer effect of the cancer immunotherapy drug by significantly enhancing the sensitivity of a subject to the cancer immunotherapy drug, but also exhibit a higher anticancer effect by activating immune cells with anticancer activity and inhibiting excessive autophagy activity through the regulation of cytokines and chemokines.

Specifically, in one embodiment of the present invention, after preparing and identifying the compound (AQTGTGKT) according to the present invention and an analog thereof, the structure of each compound was confirmed (Example 1).

In another embodiment of the present invention, as a result of administering the compound and a cancer immunotherapy drug to a cancer animal model to confirm a combined effect of a compound according to the present invention and a cancer immunotherapy drug, it was confirmed that the combination of the compound and the cancer immunotherapy drug exhibits a higher tumor inhibitory effect compared to monotherapy with each of the materials (Example 2).

In still another embodiment of the present invention, as a result of analyzing tumor tissue of a cancer animal model administered the compound and a cancer immunotherapy drug to confirm the immune cell regulatory effect of the compound according to the present invention, it was confirmed that, by the coadministration of the compound and the cancer immunotherapy drug, tumor cell death more actively occurs, vascular expansion and infiltration into tumor tissue occur, and the frequencies of T cells (particularly, cytotoxic T cells) and macrophages (particularly, M2 macrophages) in tumor tissue increase (Example 3).

In yet another embodiment of the present invention, as a result of analyzing tumor tissue in a cancer animal model administered the compound and a cancer immunotherapy drug to confirm the cytokine and chemokine regulatory effect of the compound according to the present invention, it was confirmed that the group co-administered the compound and the cancer immunotherapy drug had an increased mRNA expression level of an immunomodulatory factor in tumor tissue, compared to monotherapy with each of the materials (Example 4).

In yet another embodiment of the present invention, as a result of confirming whether the co-administration of the compound according to the present invention and a cancer immunotherapy drug affects a survival period of a cancer animal model, a group co-administered the compound and one type of cancer immunotherapy drug had an increased survival period compared to groups administered each material. Particularly, a group co-administered the compound of the present invention and two types of cancer immunotherapy drugs exhibited a longer survival period compared to the co-administered group using the compound and one type of cancer immunotherapy drug (Example 5).

The above results show that the compound according to the present invention may enhance the body's immune function by increasing the levels and activity of immune cells and immunomodulatory factors, and particularly, when used in combination with a cancer immunotherapy drug, it can further activate the function of immune cells to increase the sensitivity of a subject to a cancer immunotherapy drug and achieve a synergistic anticancer effect.

Hereinafter, the present invention will be described in detail.

The present invention provides a pharmaceutical composition for immune enhancement, which includes an oligopeptide consisting of the amino acid sequence of SEQ ID NO: 1 or an analog thereof as an active ingredient. The pharmaceutical composition for immune enhancement may further include a cancer immunotherapy drug.

In addition, the present invention provides a pharmaceutical composition for preventing or treating cancer, which includes an oligopeptide consisting of the amino acid sequence of SEQ ID NO: 1 or an analog thereof as an active ingredient.

In addition, the present invention provides a pharmaceutical composition for enhancing the anticancer effect of a cancer immunotherapy drug or for co-administration of a cancer immunotherapy drug, which includes an oligopeptide consisting of the amino acid sequence of SEQ ID NO: 1 or an analog thereof as an active ingredient.

In one embodiment of the present invention, the analog is a compound represented by the general formula X-AQTGTGKT, wherein X may be a compound represented by Chemical Formula 1 below, or a pharmaceutically acceptable salt thereof:

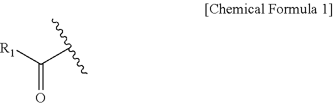

In Chemical Formula 1,
R₁ may be hydrogen, a substituted or unsubstituted phenyl group, a substituted or unsubstituted naphthyl group, or a substituted or unsubstituted C₁ to C₅ alkyl group.

Here, the phenyl group, naphthyl group and alkyl group may each have one or more functional groups independently substituted or unsubstituted with a phenyl group, a C₁ to C₃ alkoxy group, a C₁ to C₅ alkyl group, —CX₃, —OCX₃, or a morphoryl group, wherein X may be a halogen, but the present invention is not limited thereto.

Preferably, R₁ may be a compound represented by any one of Chemical Formulas 2 to 4 below:

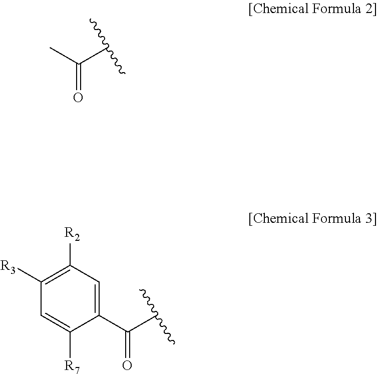

(In Chemical Formula 3,
R₂ and R₄ are each independently hydrogen, a C₁ to C₅ alkyl group, or a phenyl group; and
R₃ is hydrogen, a C₁ to C₅ alkyl group, a phenyl group, or a C₁ to C₃ alkoxy group.)

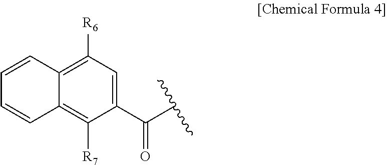

(In Chemical Formula 4,
R₆ and R₇ are each independently hydrogen or a C₁ to C₃ alkyl group.)

Most preferably, the compound according to the present invention may be a compound represented by General Formula below or a pharmaceutically acceptable salt thereof:

X-AQTGTGKT [General Formula]

(In General Formula, A is alanine, Q is glutamine, T is threonine, G is glycine, and K is lysine, and
X is not present; or is one or more selected from the group consisting of

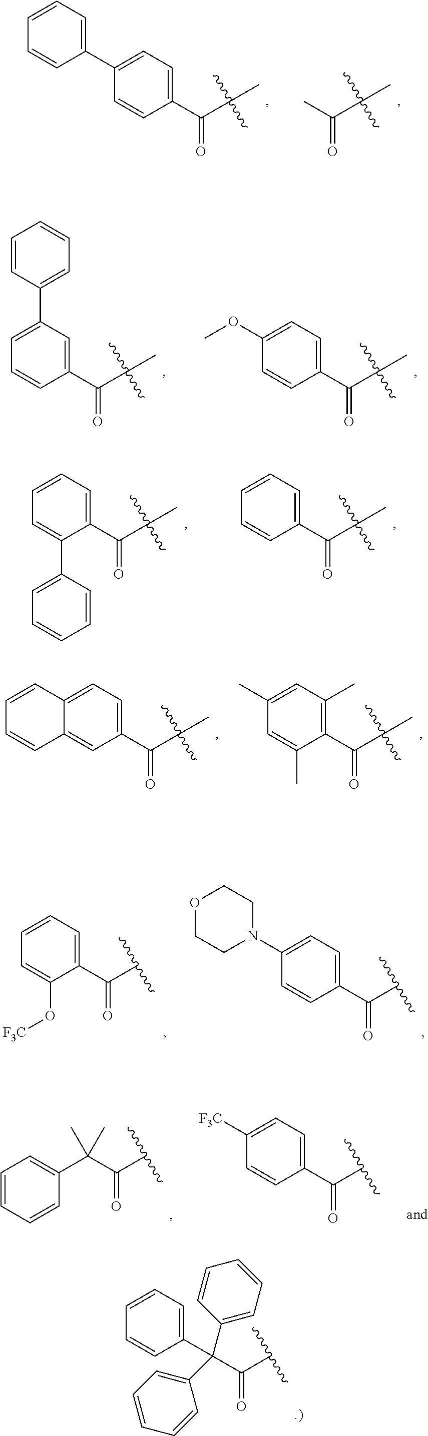

The term “oligopeptide” used herein refers to a linear molecule formed by linking amino acid residues to each other by peptide bonds. The amidated oligopeptide of the present invention may be prepared by molecular and biological methods as well as chemical synthesis methods known in the art (e.g., solid-phase synthesis techniques) (Merrifield, J. Amer. Chem. Soc. 85: 2149-54(1963); Stewart, et al., Solid Phase Peptide Synthesis, 2nd. ed., Pierce Chem. Co.: Rockford, 111(1984)). In the present invention, “analog” may refer to a material that has some differences in structure from a specific compound (e.g., differences in the type of functional group), but has a very similar structure as a whole, and similar activity and functions.

The scope of the compounds according to the present invention may also include pharmaceutically acceptable salts thereof. The term “pharmaceutically acceptable” used herein refers to a compound which is suitable for use in contact with tissue of a subject (e.g., a human) due to a reasonable benefit/risk ratio without excessive toxicity, irritation, allergic responses or other problems or complications, and is included in the scope of sound medical judgment. The pharmaceutically acceptable salt includes, for example, an acid addition salt formed by a pharmaceutically acceptable free acid and a pharmaceutically acceptable metal salt.

Specifically, examples of suitable acids may include hydrochloric acid, hydrobromic acid, sulfuric acid, nitric acid, perchloric acid, fumaric acid, maleic acid, phosphoric acid, glycolic acid, lactic acid, salicylic acid, succinic acid, p-toluenesulfonic acid, tartaric acid, acetic acid, citric acid, methanesulfonic acid, formic acid, benzoic acid, malonic acid, gluconic acid, naphthalene-2-sulfonic acid, and benzenesulfonic acid. The acid addition salt may be prepared by a conventional method, for example, by dissolving a compound in an excessive amount of acid aqueous solution, and precipitating the salt using a water-miscible organic solvent such as methanol, ethanol, acetone or acetonitrile. Alternatively, the acid addition salt may be prepared by heating equimolar amounts of compound and an acid or alcohol in water, and evaporating or drying the mixture, or suction filtering the precipitated salt.

Salts derived from suitable bases may include alkali metals such as sodium and potassium, alkaline earth metals such as magnesium, and ammonium, but the present invention is not limited thereto. The alkali metals or alkaline earth metals may be obtained by, for example, dissolving a compound in an excess alkali metal hydroxide or alkaline earth metal hydroxide solution, filtering an undissolved compound salt, and evaporating and drying the filtrate. Here, it is particularly pharmaceutically suitable to prepare a sodium, potassium or calcium salt as a metal salt, and a corresponding silver salt may be obtained by reacting an alkali metal or alkaline earth metal salt with a suitable silver salt (e.g., silver nitrate).

The scope of the compounds of the present invention may include not only pharmaceutically acceptable salts, but also all isomers, hydrates, and solvates, which can be prepared by conventional methods.

The compounds may have a non-aromatic double bond, and one or more asymmetric centers. Accordingly, they may be generated as a racemate, a racemic mixture, a single enantiomer, an individual diastereomer, a diastereomeric mixture, and a cis- or trans-isomer. All these isomeric forms are considered.

In addition, the scope of the compounds according to the present invention may include biological functional equivalents having variations in the amino acid sequence that exhibit equivalent biological activity to the compound of the present invention. These amino acid sequence variations may be made based on the relative similarity of amino acid side-chain substituents, for example, hydrophobicity, hydrophilicity, charge, and size. By analysis of the size, shape, and type of amino acid side-chain substituents, it can be seen that alanine and glycine have similar sizes; lysine is a positively charged residue; and glutamine and threonine are not changed. Accordingly, based on these considerations, alanine and glycine; and glutamine and threonine may be biologically functional equivalents.

For introduction of a variant, the hydropathic index of an amino acid may be considered. Each amino acid is assigned a hydropathic index based on its hydrophobicity and charge: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

The hydropathic index of amino acids is very important in imparting the interactive biological functions of a protein. It is a known fact that similar biological activity can be maintained only when substituted with an amino acid having a similar hydropathic index. When a variation is introduced by referring to a hydropathic index, substitutions are made between amino acids exhibiting a hydropathic index difference, preferably within ±2, more preferably within ±1, and even more preferably within ±0.5.

Meanwhile, it is also well known that substitutions between amino acids with similar hydrophilicity values result in proteins having equivalent biological activity. As disclosed in U.S. Pat. No. 4,554,101, the following hydrophilicity values are assigned to each amino acid residue: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

When a variant is introduced by referring to a hydrophilicity value, substitutions are made between amino acids which exhibit a hydrophilicity value difference, preferably within ±2, more preferably within ±1, and even more preferably within ±0.5.

Amino acid exchanges in proteins, which do not overall change the activity of a molecule are known in the art (H. Neurath, R. L. Hill, The Proteins, Academic Press, New York, 1979). The most common exchanges are the exchanges between amino acid residues, such as Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

Considering the above-described variants having biologically equivalent activity, the protein of the amino acid sequence (AQTGTGKT) of the compound represented by General Formula of the present invention is interpreted to also include a sequence exhibiting substantial identity to the above sequence. The substantial identity refers to a sequence having at least 62.5% homology, more preferably, 75% or more homology, and most preferably, 87.5% or more homology, when the sequence of the present invention is aligned to correspond to another sequence as much as possible, and the aligned sequence is analyzed using an algorithm conventionally used in the art. An alignment method for sequence comparison is known in the art.

In the present invention, “immune enhancement” refers to the function of regulating the body's immune response to an appropriate level, and specifically includes not only a function of activating an immune response for protecting the body from an external factor (bacteria, fungi, or viruses) or a tumor, but also a function of suppressing a hypersensitive immune response caused by allergens or autoantigens (autoimmune reaction).

More specifically, the oligopeptide or an analog thereof may satisfy one or more characteristics selected from the group consisting of the following:
(a) increasing an immune response against one or more selected from the group consisting of tumor antigens (cancer cell-specific antigens), bacterial infections, and viral infections;
(b) suppressing immune hypersensitivity;
(c) regulating the level or activity of one or more selected from the group consisting of CXCL10, IFNγ, TNFα, CCL5, CXCL9, CXCL11, IL-1α, IL-1β, IL-6, and IL-12; and
(d) increasing the level or activity of one or more selected from the group consisting of cytotoxic T cells and M2 macrophages.

In (c), the regulating of the level or activity encompasses both the promotion (increase) and suppression (decrease) of the level or activity. In addition, the above immunomodulatory factors are only non-limiting examples, and the oligopeptide or an analog thereof according to the present invention may regulate the level or activity of various immunomodulatory factors as well as the above immunomodulatory factors.

The immune enhancing function mediated by the compound of the present invention may be achieved by regulating the activity and level of various immune cells, cytokines, and chemokines. For example, the compound of the present invention may enhance immune responses to the invasion of pathogenic factors or abnormal cells such as cancer cells and suppress excessive immune responses by regulating (increasing or suppressing) the level or activity of antigen-presenting cells, natural killer (NK) cells, T cells (cytotoxic T cells and regulatory T cells), B cells, macrophages (M1 and M2 macrophages), and dendritic cells, resulting in the suppression of hypersensitive immune responses to autoantigens/allergens or inflammation.

Particularly, the compound according to the present invention may exhibit an excellent anticancer effect through an immunomodulatory function, and satisfy one or more characteristics selected from the group consisting of the following, but the present invention is not limited thereto:
(a) increasing the level or activity of tumor-suppressive immune cells;
(b) suppressing the level or activity of immunosuppressive immune cells; and
(c) reducing an autophagy level.

In another embodiment of the present invention, the tumor-suppressive immune cells may be one or more selected from the group consisting of CD3⁺ T cells, CD8⁺ T cells, CD19⁺ B cells, CD11b⁺ dendritic cells, CD49b⁺ NK cells, and CD68⁺/CD206⁻ M1 macrophages, but the present invention is not limited thereto.

In still another embodiment of the present invention, the immunosuppressive immune cells may be one or more selected from the group consisting of Foxp3⁺ Treg cells and CD68⁺/CD206⁺ tumor-associated macrophages, but the present invention is not limited thereto.

Preferably, the immune enhancing function of the compound according to the present invention may be achieved by suppressing an excessive autophagy level. In the present invention, “autophagy” used herein is an intracellular decomposition mechanism that occurs under various stress conditions, including cell organelle damage, the presence of abnormal proteins, and nutrient deficiency, and refers to the natural decomposition of unnecessary or non-functional cellular components. Autophagy may affect an immune response in the body, and particularly, is known to be involved in both tumor suppression and promotion, so research is needed to discover the exact mechanism of autophagy in cancer. The compound according to the present invention may inhibit autophagy, prevent excessive activation of immune cells, and enhance the anticancer effect of cancer immunotherapy drugs on cancer cells.

In addition, the compound of the present invention may increase the sensitivity of a subject to a cancer immunotherapy drug and maximize the anticancer effect of the cancer immunotherapy drug by regulating the activity and level of immune cells and immunomodulatory factors involved in immune responses. Therefore, the compound of the present invention may enhance the anticancer effect of a cancer immunotherapy drug and reduce the side effects thereof.

The term “coadministration” used herein may be achieved by simultaneously, sequentially, or separately administering separate components of a treatment regimen. The combination therapy effect is obtained by simultaneously, sequentially, or alternately administering two or more drugs at regular or undetermined intervals. Combination therapy is not limited to the above, and may be defined as something that is therapeutically superior to the efficacy that can be obtained by administering one or the other components of the combination therapy at a conventional dose, when the efficacy is measured, for example, through the degree of response, the speed of response, the time to disease progression or a survival period, and can provide a synergistic effect.

The term “anticancer agent” used herein is used as a general term for a material used for the treatment of malignant tumors. Most anticancer drugs are drugs that mainly inhibit nucleic acid synthesis or exhibit anticancer activity by interfering with various types of metabolic pathways of cancer cells. Anticancer drugs that are currently used in cancer treatment are classified into six categories according to their biochemical mechanisms of action: alkylating agents, antimetabolites, antibiotics, vinca alkaloids, hormonal agents, and the others, but the anticancer agent according to the present invention may not be included in the above categories.

Preferably, the anticancer agent according to the present invention is a “cancer immunotherapy drug.” Cancer immunotherapy is a cancer treatment that activates the immune system of the human body to fight cancer cells, and a cancer immunotherapy drug exhibits an anticancer effect by enhancing the specificity, memory, and adaptiveness of the immune system. That is, cancer immunotherapy has fewer side effects by precisely attacking only cancer cells using the immune system of the human body, and using the memory and adaptiveness of the immune system, a patient that responds to a cancer immunotherapy drug can exhibit a sustained anticancer effect. Preferably, the cancer immunotherapy drug may be one or more selected from the group consisting of an immune checkpoint inhibitor, a costimulatory molecule agonist, a cytokine therapeutic, a CAR-T cell therapeutic, and an autologous CD8⁺ T immune cell therapeutic, but the present invention is not limited thereto.

Immune checkpoint inhibitors refer to agents that suppresses an immune checkpoint involved in the immune evasion mechanism of cancer cells. Some cancer cells evade immunity by utilizing the immune checkpoints of immune cells, and immune checkpoint inhibitors block immune evasion signals by binding to the binding sites of cancer cells and T cells, preventing the formation of an immunological synapse. Therefore, T cells that are not hindered by immune evasion have a mechanism to destroy cancer cells. The immune checkpoint inhibitor may be one or more selected from the group consisting of a PD-L1 inhibitor, a PD-1 inhibitor, a CTLA-4 inhibitor, a LAG3 inhibitor, a TIM3 inhibitor, a 4-1BB inhibitor, a LAG-3 inhibitor, a B7-H4 inhibitor, an HVEM inhibitor, a TIM4 inhibitor, a GAL9 inhibitor, a VISTA inhibitor, a KIR inhibitor, a TIGIT inhibitor, and a BTLA inhibitor, but the present invention is not limited thereto. In another embodiment of the present invention, the costimulatory molecule agonist may be one or more selected from the group consisting of a 4-1BB inhibitor, and an OX40 inhibitor, but the present invention is not limited thereto. The “inhibitor” is sufficient as long as it can inhibit the activity or level of a target, and is not limited to a specific type. However, the inhibitor is preferably a compound or antibody (i.e., an antibody specifically binding to a target). As the cancer immunotherapy drug according to the present invention, a current commercially available cancer immunotherapy drug, e.g., atezolizumab, avelumab, dostarlimab, durvalumab, ipilimumab, nivolumab, or pembrolizumab may be applied.

In addition, cytokine therapeutics refer to therapeutics for regulating the activity or level of immune cells by cytokine administration, resulting in regulating an immune response. The cytokine therapeutic according to the present invention may be selected from IL-2, IFNγ, IFNα, TNFα, IFNα, and IL-12, but the present invention is not limited thereto.

The pharmaceutical composition for coadministering a cancer immunotherapy drug according to the present invention may enhance the anticancer effect of a cancer immunotherapy drug and reduce side effects. This is because the dosage of a cancer immunotherapy drug with side effects can be minimized through appropriate combination therapy. Here, the “enhancing an anticancer effect” means increasing all effects capable of consequently enhancing the function of a cancer immunotherapy drug includes the enhancement of the anticancer effect of a cancer immunotherapy drug, such as the suppression of tumor growth, the suppression of tumor metastasis, and the suppression of tumor recurrence, results from suppressing the resistance or tolerance of cancer cells to a cancer immunotherapy drug. The compound according to the present invention preferably enhances the sensitivity of a subject to a cancer immunotherapy drug.

In the present invention, the compound or a composition including the same may be simultaneously, separately, or sequentially administered with a cancer immunotherapy drug, and even when administered sequentially with an anticancer agent, the administration order is not limited. However, a dosage regimen may be appropriately adjusted according to the type of cancer, the type of anticancer agent, and a patient's condition.

The content of the compound or anticancer agent in the composition of the present invention can be appropriately adjusted according to the symptoms of a disease, the degree of the progression of the symptoms, or a patient's condition, and for example, the content may be 0.0001 to 99.9 wt %, or 0.001 to 50 wt % based on the total weight of the composition, but the present invention is not limited thereto. The content ratio is a value based on a dry weight from which a solvent is removed.

For example, in the composition according to the present invention, the weight ratio of the cancer immunotherapy drug:the oligopeptide or an analog thereof may be 1:0.1 to 10, but the present invention is not limited thereto. Specifically, the weight ratio of the cancer immunotherapy drug:the oligopeptide or an analog thereof in the composition of the present invention may be 1:0.1 to 10, 1:0.1 to 5, 1:0.1 to 3, 1:0.1 to 2.5, 1:0.1 to 2, 1:0.5 to 5, 1:1 to 5, or 1:1 to 3, but the present invention is not limited thereto.

In addition, the composition according to the present invention may be in the form of a mixture in which the compound and the anticancer agent are mixed and may be in a form for simultaneous administration of the compound and the anticancer agent.

In addition, the composition according to the present invention may be simultaneously, separately, or sequentially administered after separately preparing the compound and the anticancer agent. In this case, the composition may be a pharmaceutical composition for simultaneous or sequential coadministration, which includes a first pharmaceutical composition including a pharmaceutically effective amount of the compound as an active ingredient; and a second pharmaceutical composition including a pharmaceutically effective amount of the anticancer agent as an active ingredient. Here, for sequential administration, the administration sequence is not limited, and a dosage regimen may be appropriately adjusted according to a patient's condition.

That is, when the pharmaceutical composition is a pharmaceutical composition for sequential coadministration, to prepare the composition, the compound (first component) may be administered first and then the anticancer agent (second component) may be administered, and the reverse order is also possible.

In addition, the composition according to the present invention may include two or more types of cancer immunotherapy drugs. The present inventors confirmed that, when the compound according to the present invention is used in combination with two types of cancer immunotherapy drugs (anti-PD-1 and anti-CTLA4) according to a specific example (triple combination), it exhibits not only a greater anticancer effect than the single use of the compound according to the present invention or each drug, but also a significantly increased anticancer effect compared to the dual use of the compound of the present invention and each cancer immunotherapy drug. Therefore, when the compound according to the present invention is used in a triple combination with two or more cancer immunotherapy drugs, better cancer prevention and treatment effects may be achieved.

Accordingly, when the composition according to the present invention includes the compound or a pharmaceutically acceptable salt thereof; and two types of cancer immunotherapy drugs (e.g., a first cancer immunotherapy drug and a second cancer immunotherapy drug), each of the two types of cancer immunotherapy drugs may be included at a weight ratio of 1:0.1 to 10 (the first or second cancer immunotherapy drug:the oligopeptide or an analog thereof) compared to the compound (the oligopeptide of the present invention or an analog thereof). Preferably, the two types of cancer immunotherapy drugs may be immune checkpoint inhibitors, and more preferably are two or more selected from the group consisting of a PD-L1 inhibitor, a PD-1 inhibitor, a CTLA-4 inhibitor, a LAG3 inhibitor, a TIM3 inhibitor, a 4-1BB inhibitor, a LAG-3 inhibitor, a B7-H4 inhibitor, an HVEM inhibitor, a TIM4 inhibitor, a GAL9 inhibitor, a VISTA inhibitor, a KIR inhibitor, a TIGIT inhibitor, and a BTLA inhibitor. Most preferably, the two types of cancer immunotherapy drugs may be selected from a PD-L1 inhibitor, a PD-1 inhibitor, and a CTLA-4 inhibitor.

The compound according to the present invention may be used in the prevention and/or treatment of cancer. The term “cancer” used herein is characterized by uncontrolled cell growth, and due to this abnormal cell growth, a cell mass called a tumor is formed and infiltrates surrounding tissue, which may, in severe cases, metastasize to other organs in the body. Academically, it is also called a neoplasm. Cancer is an intractable chronic disease that cannot be fundamentally cured even when treated with surgery, radiation, and chemotherapy in many cases, is painful for a patient, and ultimately leads to death. There are many causes of cancer, which are divided into internal and external factor. Although the exact mechanism by which normal cells are transformed into cancer cells has not be identified, it is known that a significant number of cancer types are caused by external factors such as environmental factors. The internal factors include genetic factors and immunological factors, and the external factors include chemicals, radiation, and viruses. Genes involved in the development of cancer include oncogenes and tumor suppressor genes, and cancer occurs when the balance between these genes is broken by the internal or external factors described above.

The cancer may be solid cancer or blood cancer, and non-limiting examples thereof may be one or more selected from the group consisting of squamous cell carcinoma, lung cancer, adenocarcinoma of the lung, peritoneal cancer, skin cancer, skin or intraocular melanoma, rectal cancer, anal cancer, esophageal cancer, small intestine cancer, endocrine adenocarcinoma, parathyroid cancer, adrenal cancer, soft tissue sarcoma, urethral cancer, blood cancer, liver cancer, gastrointestinal cancer, pancreatic cancer, glioblastoma, cervical cancer, ovarian cancer, bladder cancer, hepatocellular carcinoma, breast cancer, colon cancer, colorectal cancer, endometrial cancer, uterine cancer, salivary gland cancer, kidney cancer, prostate cancer, vulva cancer, thyroid cancer, head and neck cancer, and brain cancer, and more specifically, cancer selected from lung cancer, breast cancer, blood cancer, colorectal cancer, pancreatic cancer, and a combination thereof.

In the present invention, the cancer may be cancer accompanying one or more selected from the group consisting of a KRAS mutation, and an EGFR mutation. That is, the compound according to the present invention or a pharmaceutically acceptable salt thereof may exhibit an excellent immunomodulatory or anticancer effect particularly in cancer accompanying the mutation.

Alternatively, the cancer according to the present invention may accompany a mutation in which the level or activity of a KRAS mutation is excessively increased. For example, the cancer of the present invention may involve a KRAS G12D or V8M mutation.

In addition, the cancer according to the present invention may be cancer accompanying mutations such as an EGFR mutation (an increase or decrease in expression/activity), a microsatellite instability-high (MSS-high) mutation, a microsatellite instability-low (MSI-low) mutation, and/or a microsatellite stability (MSS) mutation. The MSS-high may mean ‘high microsatellite instability,’ MSI-low may mean ‘low microsatellite instability,’ and MSS may mean ‘no instability.’

In addition, the cancer according to the present invention may be characterized by an increased autophagy level or activity, compared to a normal level. That is, the composition according to the present invention may exhibit a higher immunomodulatory effect or anticancer effect in a subject with an increased autophagy level.

Meanwhile, the cancer may be cancer cells in which the level (i.e., expression) or activity of CAGE increases. “Cancer-associated gene protein (CAGE; or cancer/testis antigen)” is known to be testis-specifically expressed in a normal person, but expressed in various types of cancer tissue in a cancer patient. CAGE is a known protein, and its specific data may be found in the public protein database UniProt under Registration No. Q8TC20.

In addition, the present invention provides a kit for immune enhancement, a kit for preventing or treating cancer, a kit for enhancing the anticancer effect of a cancer immunotherapy drug, and/or a kit for coadministering a cancer immunotherapy drug, each of which includes a compound according to the present invention (AQTGTGKT oligopeptide or a salt thereof) or a pharmaceutically acceptable salt thereof.

In addition to the compound or anti-cancer agent, the kit according to the present invention may include other components, compositions, solutions, and devices, which are conventionally needed for the prevention or treatment of cancer without limitation, and particularly, include instructions for proper use and storage of the compound of the present invention.

In the present invention, “prevention” refers to all actions that suppress or delay the onset of a target disease, “treatment” refers to all actions that improve or beneficially change the symptoms of target diseases and associated metabolic disorders through the administration of the pharmaceutical composition according to the present invention, and “improvement” refers to all actions that reduce parameters, for example, the degree of symptoms related to target diseases by the administration of the composition according to the present invention.

The cancer prevention and/or treatment effect includes not only an effect of inhibiting the growth of cancer cells but also an effect of inhibiting the worsening of cancer due to migration, invasion, or metastasis.

The term “subject” used herein means a subject in need of prevention or treatment of a disease. For example, the subject may include mammals, including humans, non-human primates, mice, dogs, cats, horses, sheep, and cattle.

Meanwhile, the pharmaceutical composition according to the present invention may further include appropriate carriers, excipients, and/or diluents, which are conventionally used to prepare pharmaceutical compositions, in addition to an active ingredient. In addition, according to a conventional method, it may be formulated and used in the form of oral dosage forms such as powders, granules, tablets, capsules, suspensions, emulsions, syrups, aerosols, external preparations, suppositories, and sterile injection solutions.

Carriers, excipients, and diluents, which can be included in the composition, may include lactose, dextrose, sucrose, sorbitol, mannitol, xylitol, maltitol, starch, gum acacia, alginate, gelatin, calcium phosphate, calcium silicate, cellulose, methyl cellulose, microcrystalline cellulose, methyl cellulose, microcrystalline cellulose, polyvinyl pyrrolidone, water, methylhydroxy benzoate, propylhydroxy benzoate, talc, magnesium stearate, and mineral oil. When the composition is prepared, it can be formulated using commonly used diluents or excipients, such as a filler, an extender, a binder, a wetting agent, a disintegrant, and a surfactant.

The “administration” used herein refers to providing the given composition of the present invention to a subject by any appropriate method.

The pharmaceutical composition of the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including the type of a patient's disease, severity, drug activity, sensitivity to a drug, administration time, an administration route and an excretion rate, the duration of treatment and drugs simultaneously used, and other parameters well known in the medical field. A preferable dose of the pharmaceutical composition of the present invention may be selected according to a subject's condition and body weight, the severity of a disease, a drug type, an administration route and administration duration. As a specific example, the pharmaceutical composition may be administered once or several times a day at a dose of 0.001 to 1000 mg/kg, 0.01 to 100 mg/kg, 0.01 to 10 mg/kg, 0.1 to 10 mg/kg, or 0.1 to 1 mg/kg.

Preferably, the composition of the present invention may be administered to a subject such that the cancer immunotherapy drug is administered at a concentration of 0.1 to 50 mg/kg, 0.1 to 30 mg/kg, 0.1 to 10 mg/kg, 0.1 to 7 mg/kg, 0.1 to 5 mg/kg, 0.1 to 3 mg/kg, 1 to 10 mg/kg, 1 to 7 mg/kg, 1 to 5 mg/kg, or 1 to 3 mg/kg. Alternatively, the composition of the present invention may be administered to a subject such that the oligopeptide or an analog thereof is administered at a concentration of 0.01 to 500 mg/kg, 0.01 to 400 mg/kg, 0.01 to 300 mg/kg, 0.01 to 200 mg/kg, 0.01 to 100 mg/kg, 0.05 to 100 mg/kg, 0.1 to 100 mg/kg, 0.1 to 50 mg/kg, 0.1 to 40 mg/kg, 0.1 to 30 mg/kg, 0.1 to 20 mg/kg, 0.1 to 15 mg/kg, 0.1 to 12 mg/kg, 0.1 to 10 mg/kg, 1 to 20 mg/kg, 1 to 15 mg/kg, 1 to 12 mg/kg, 1 to 10 mg/kg, 5 to 20 mg/kg, 5 to 15 mg/kg, 5 to 12 mg/kg, or 5 to 10 mg/kg. In addition, when the cancer immunotherapy drug is used in combination with the compound of the present invention, the cancer immunotherapy drug and the compound may each be independently administered every 1 to 10 days, 1 to 8 days, 1 to 6 days, 1 to 4 days, 1 to 3 days, or 1 to 2 days.

Considering all of the above factors, it is important to administer an amount that can achieve the maximum effect with the minimum amount without side effects, which may be determined by one of ordinary skill in the art. Specifically, the effective amount of the pharmaceutical composition according to the present invention may vary according to a patient's age, sex, condition, and weight, the absorption of an active ingredient in the body, an inactivation rate, an excretion rate, a disease type, or a co-administered drug.

The pharmaceutical composition of the present invention may be administered to a subject via various routes. All administration routes may be considered, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration. A daily dose may be administered once or several times a day.

Terms and words used in the specification and claims should not be construed as being limited to general or dictionary terms meanings, and should be interpreted with the meaning and concept in accordance with the technical idea of the present invention based on the principle that the inventors have appropriately defined the concepts of terms in order to explain the invention in the best way.

Terms and words used in the specification and claims should not be construed as being limited to general or dictionary terms meanings, and should be interpreted with the meaning and concept in accordance with the technical idea of the present invention based on the principle that the inventors have appropriately defined the concepts of terms in order to explain the invention in the best way.

Hereinafter, preferred preparation examples and experimental examples will be presented to better explain the present invention. However, the following preparation examples and experimental examples are merely provided to better explain the present invention, and the present invention is not limited to the following preparation examples.

### EXAMPLES

### Example 1: Preparation of AQTGTGKT Analogs

### 1.1. Overview of Reactions

All reactions were performed using commercially available materials and reagents without additional reactions unless otherwise stated. The reactions were monitored using thin layer chromatography (TLC) on a silica gel plate (Keiselgel 60 F254, Merck) and/or ultra-performance liquid chromatography (UPLC). The visualization of spots on the TLC plate was achieved by UV light, and staining the TLC plate with potassium permanganate and/or ninhydrin and carbonizing the plate using a heat gun. All products were characterized using ¹H NMR and/or UPLC-MS.

### 1.2. Synthesis of Boc/OBn-TG

First, TG whose functional group was protected with benzyl was synthesized according to Reaction Scheme 1 below. Hereinafter, in each Reaction Scheme, a compound will be referred to as Compound n according to the Arabic number (n) indicated below the Compound.

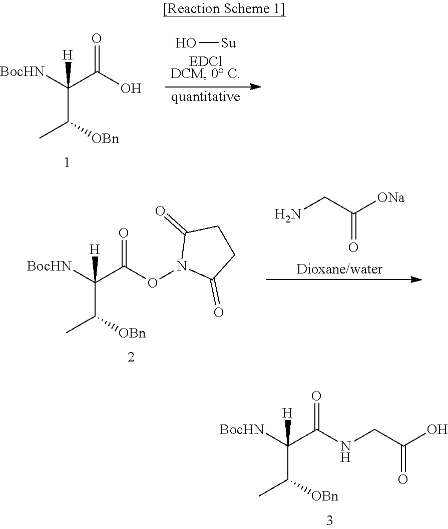

Specifically, BocThr(OBn)OH (Compound 1; 25.0 g, 80.8 mmol, 1.0 eq) and NOSu (9.77 g, 84.8 mmol, 1.05 eq) were dissolved in dichloromethane (150 mL). The mixture was cooled to 0° C. and placed in an inert gas. Afterward, 1-(3-dimethylaminopropyl)-3-ethylcarbodiimide hydrochloride (16.3 g, 84.8 mmol, 1.05 eq) was added to the mixture. The mixture was warmed to room temperature and stirred for 20 hours. Subsequently, the mixture was washed with NH₄Cl (saturated aqueous) and the phases were separated. The organic layer was dehydrated over MgSO₄ and concentrated under reduced pressure, thereby obtaining Compound 2 as a light-yellow oil (35.7 g, >100% yield, assuming a quantitative yield).

Compound 2, BocThr(OBn)OSu (32.8 g, 80.8 mmol, 1.0 eq), was dissolved in 1,4-dioxane (200 mL), and a glycine sodium salt hydrate solution in distilled water (100 mL) was added in one portion. After stirring at room temperature for 6 hours, the mixture was fractionated with ethyl acetate and citric acid (saturated aqueous). The organic layer was dehydrated over MgSO₄, filtered, and concentrated under reduced pressure. A crude material was purified on a C18 (400 g) column with a gradient of 30 to 70% acetonitrile (0.1% formic acid) in a water (0.1% formic acid) eluent. The desired fractions were combined and fractionated with ethyl acetate and NaHCO₃ (saturated aqueous). The organic layer was dehydrated over MgSO₄, filtered, and concentrated under reduced pressure, thereby obtaining Compound 3 as a light-yellow gum (21.9 g, 74% yield).

### 1.3. Synthesis of CBz/OBn/CO₂Bn-KT

KT whose OH functional group was protected with benzyl was synthesized according to Reaction Scheme 2 below.

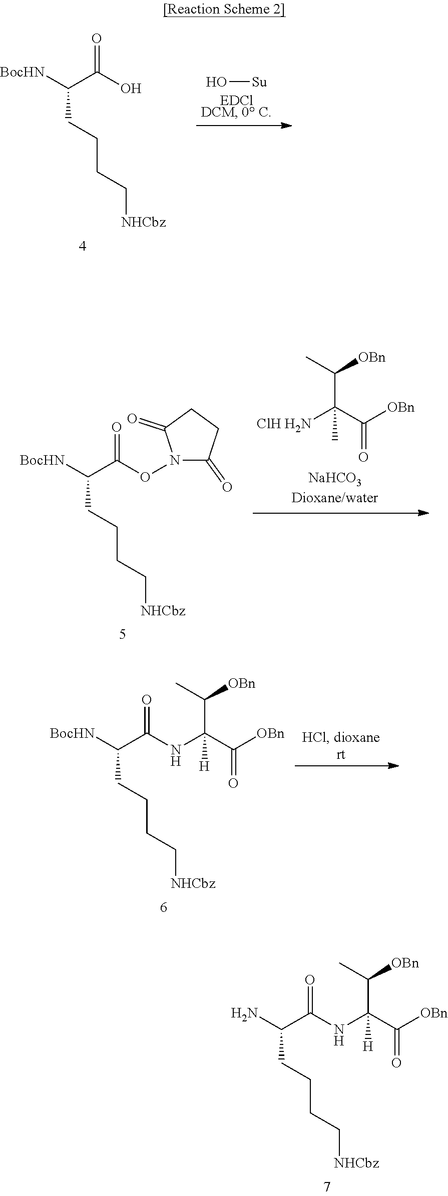

Specifically, BocLys(CBz)OH (Compound 4; 27.0 g, 70.9 mmol, 1.0 eq) and NOSu (9.80 g, 85.1 mmol, 1.2 eq) were dissolved in dichloromethane (128 mL). The mixture was cooled to 0° C., and placed in an inert gas. Afterward, 1-(3-dimethylaminopropyl)-3-ethylcarbodiimide hydrochloride (16.3 g, 85.1 mmol, 1.05 eq) was added. The mixture was warmed to room temperature and stirred for 20 hours. Subsequently, the mixture was washed with NH₄Cl (saturated aqueous) and the phases were separated. The organic layer was dehydrated over MgSO₄ and concentrated under reduced pressure, thereby obtaining Compound 5 as a light-yellow oil (36.7 g, >100% yield, assuming a quantitative yield).

Subsequently, Compound 5 (BocLys(Cbz)OSu; 36.7 g, 70.9 mmol, 1.0 eq) and Thr(OBn)OBn·HCl (25.0 g, 74.4 mmol, 1.05 eq) were dissolved in 1,4-dioxane (477 mL) at room temperature. A NaHCO₃ (6.85 g, 81.5 mmol, 1.15 eq) solution in distilled water (326 mL) was added to the above mixture. Afterward, the resulting mixture was stirred at room temperature for 20 minutes. The reaction mixture was diluted with ethyl acetate and washed with 10% citric acid (aqueous) and brine. The organic layer was dehydrated over Na₂SO₄, filtered and concentrated under reduced pressure, thereby obtaining Compound 6 as a yellow oily solid (55.9 g, >100% yield, assuming a quantitative yield).

Finally, Compound 6 (BocLys(Cbz)Thr(OBn)OBn; 55.9 g, 70.9 mmol, 1.00 eq) was dissolved in 1,4-dioxane (360 mL), and 4N HCl in 1,4-dioxane (177 mL) was added. The mixture was stirred overnight at room temperature. Afterward, a saturated aqueous solution of NaHCO₃ was added until the pH value reached 8. The solution was extracted with ethyl acetate, the resulting organic solution was dehydrated over Na₂SO₄, filtered and concentrated under reduced pressure, thereby obtaining Compound 7 as a yellow oily solid (38.7 g, 97% yield).

### 1.4. Synthesis of CBz/OBn/OBn/CO₂Bn-TGKT

The TG and KT synthesized above in 1.2. and 1.3. were combined according to Reaction Scheme 3 below, thereby synthesizing TGKT.

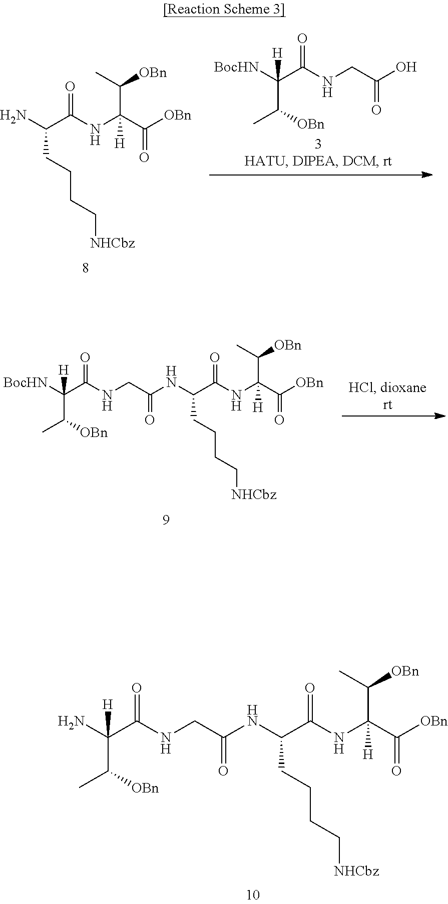 [SMILES: C[C@@H](OCc1ccccc1)[C@H](NC(=O)[C@@H](N)CCCCNC(=O)OCc1ccccc1)C(=O)OCc1ccccc1 ; C[C@@H](OCc1ccccc1)[C@H](NC(=O)OC(C)(C)C)C(=O)NCC(=O)O ; C[C@@H](OCc1ccccc1)[C@H](NC(=O)OC(C)(C)C)C(=O)NCC(=O)N[C@@H](CCCCNC(=O)OCc1ccccc1)C(=O)N[C@H](C(=O)OCc1ccccc1)[C@@H](C)OCc1ccccc1 ; C[C@@H](OCc1ccccc1)[C@H](N)C(=O)NCC(=O)N[C@@H](CCCCNC(=O)OCc1ccccc1)C(=O)N[C@H](C(=O)OCc1ccccc1)[C@@H](C)OCc1ccccc1]

More specifically, N,N-diisopropylethylamine (5.90 mL, 33.7 mmol, 2.2 eq) was added to a solution of BocThr(OBn)GlyOH (Compound 3; 5.61 g, 15.3 mmol, 1.00 eq) and Compound 8 (Lys (Cbz)Thr(OBn) OBn; 10.0 g, 15.3 mmol, 1.0 eq) in dichloromethane (50 mL). The mixture was stirred under an insert gas at room temperature, and HATU (7.00 g, 18.4 mmol, 1.20 eq) was added. The resulting mixture was stirred for 2 hours, washed with NH₄Cl (saturated aqueous), and then washed with NaHCO₃ (saturated aqueous). The organic layer was dehydrated over Na₂SO₄, filtered and concentrated under reduced pressure, thereby obtaining Compound 9 as a light orange oily solid (25.0 g, >100% yield, assuming a quantitative yield).

The obtained BocThr(OBn)GlyLys(Cbz)Thr(OBn)OBn (Compound 9; 13.9 g obtained from the previous step, 15.3 mmol, 1.0 eq) was dissolved in 1,4-dioxane (150 mL) at room temperature under nitrogen. 4N HCl in 1,4-dioxane (20 mL) was added to the above solution. The mixture was stirred for 20 hours at room temperature. The mixture was concentrated under reduced pressure and purified on a C18 (400 g) column with a 20% acetonitrile (0.1% formic acid) in a water (0.1% formic acid) eluent. The desired fractions were combined and freeze-dried. The resulting powder was dissolved in NaHCO₃ (saturated aqueous) and dichloromethane and stirred for 15 minutes. After layer separation, the organic layer was dehydrated over Na₂SO₄, filtered and concentrated under reduced pressure, thereby obtaining Compound 10 as a colorless gum (10.9 g, 88% yield).

### 1.5. Synthesis of CBz/OBn/OBn/OBn/CO₂Bn-TGTGKT

The TG (Compound 3) and TGKT (Compound 10) synthesized above in 1.2. and 1.4. were combined according to Reaction Scheme 4 below, thereby synthesizing TGTGKT protected with benzyl.

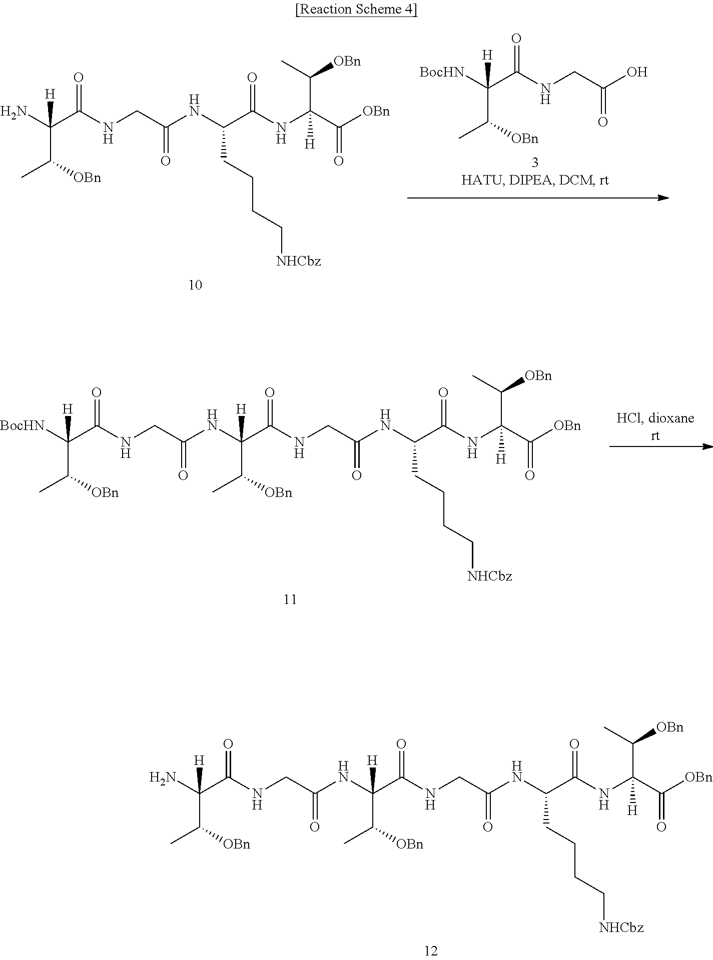

More specifically, N,N-diisopropylethylamine (5.10 mL, 29.3 mmol, 2.2 eq) was added to a solution of BocThr(OBn)GlyOH (Compound 3; 5.10 g, 14.0 mmol, 1.05 eq) and BocThr(OBn)GlyLys(Cbz)Thr(OBn) OBn (Compound 10; 10.8 g, 13.3 mmol, 1.0 eq) in dichloromethane (100 mL). The mixture was stirred under an inert gas at room temperature and HATU (5.60 g, 14.7 mmol, 1.1 eq) was added. The resulting mixture was stirred for 2 hours and washed with NH₄Cl (saturated aqueous) and NaHCO₃ (saturated aqueous). The organic layer was concentrated under reduced pressure, thereby obtaining Compound 11 as a light-yellow gum (21.4 g, >100% yield, assuming a quantitative yield).

Subsequently, Compound 11 (BocThr(OBn)GlyThr(OBn)GlyLys(Cbz)Thr (OBn)OBn; 15.4 g obtained from the previous step, 13.3 mmol, 1.00 eq) obtained above was dissolved in 1,4-dioxane (150 mL) at room temperature under nitrogen. 4N HCl in 1,4-dioxane (50 mL) was added to the above solution, and the mixture was stirred for 5 hours at room temperature. The mixture was concentrated under reduced pressure and purified on a C18 (120 g) column using 20% acetonitrile (0.1% formic acid) in a water (0.1% formic acid) eluent. The desired fractions were combined, concentrated to half volume, and then fractionated with NaHCO₃ (saturated aqueous) and ethyl acetate. After layer separation, the organic layer was dehydrated over Na₂SO₄, filtered and concentrated under reduced pressure, thereby obtaining Compound 12 as an off-white solid (14.6 g, >100% yield, assuming a quantitative yield).

### 1.6. Synthesis of CBz/OBn/OBn/OBn/CO₂Bn-QTGTGKT

Compound 14 (QTGTGKT) was synthesized by additionally combining Q and Compound 12 (TGTGKT) synthesized in 1.5 according to Reaction Scheme 5 below.

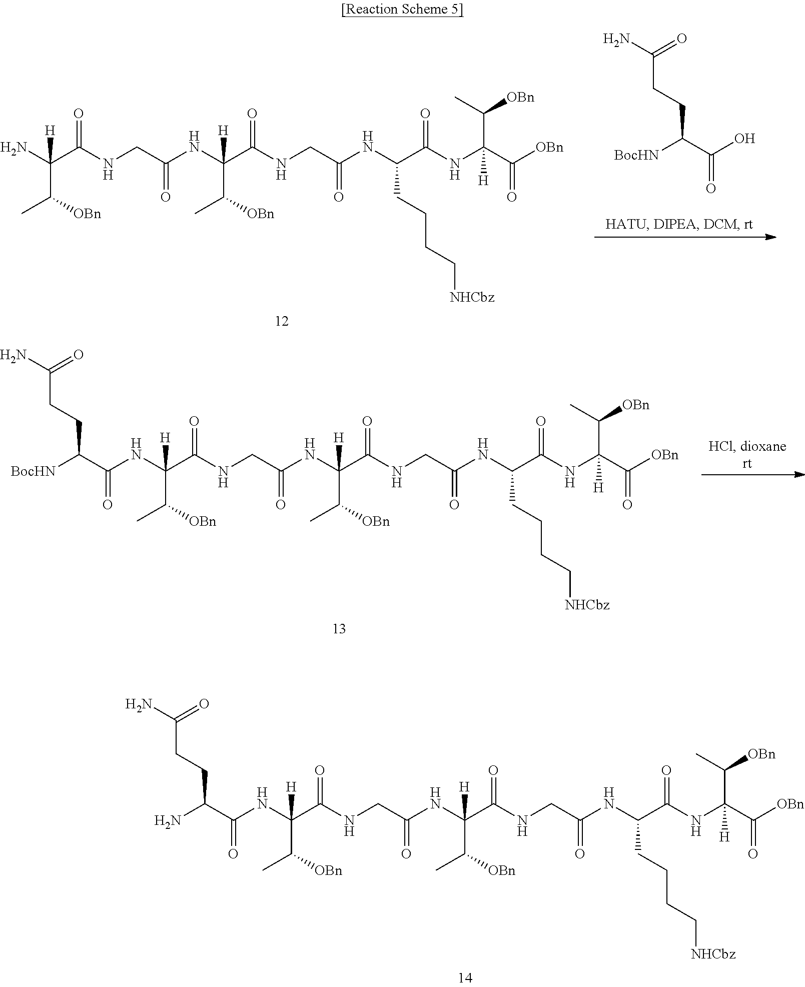

More specifically, N,N-diisopropylethylamine (4.60 mL, 26.4 mmol, 2.2 eq) was added to a solution of Thr(OBn)GlyThr(OBn)GlyLys(Cbz)Thr(OBn)OBn (Compound 12; 12.7 g, 12.0 mmol, 1.0 eq) and BocGlnOH (3.25 g, 13.2 mmol, 1.1 eq) in ethyl acetate (150 mL) and N,N-dimethylformamide (25 mL). The mixture was stirred under an inert gas at room temperature, and HATU (5.47 g, 14.4 mmol, 1.20 eq) was added. The resulting mixture was stirred for 1 hour, and then washed with NH₄Cl (saturated aqueous). The organic layer was additionally extracted with dichloromethane. Subsequently, the organic layers were combined, and then the resulting mixture was concentrated under reduced pressure. A crude material was purified on a C18 (400 g) column with a gradient of 20 to 100% acetonitrile (0.1% formic acid) in water (0.1% formic acid). The desired fractions were combined and then fractionated with ethyl acetate and NaHCO₃ (saturated aqueous). The organic layer was concentrated, and the remaining water was removed by a freeze-drying process. A total of 12.9 g (89% overall yield) of Compound 13 was obtained from the combined fractions.

Compound 13 (BocGlnThr(OBn)GlyThr(OBn)GlyLys(Cbz)Thr(OBn) OBn; 7.00 g, 5.40 mmol, 1.00 eq) was dissolved in 1,4-dioxane (150 mL) at room temperature under nitrogen. 4N HCl in 1,4-dioxane (43.5 mL) was added to the above solution. The mixture was stirred for 20 hours at room temperature. The mixture was concentrated under reduced pressure and freeze-dried using a water/acetonitrile (2/1) solution. Finally, Compound 14 as a light-yellow powder (6.36 g, 96% yield) was isolated.

### 1.7. Synthesis of AQTGTGKT Analogs

Six types of analogs excluding 4-PhPh-AQTGTGKT were synthesized by combining Compound 14 as the final product of Reaction Scheme 5 and Compound 17n as a product of Reaction Scheme 6 below according to Reaction Scheme 7 below.

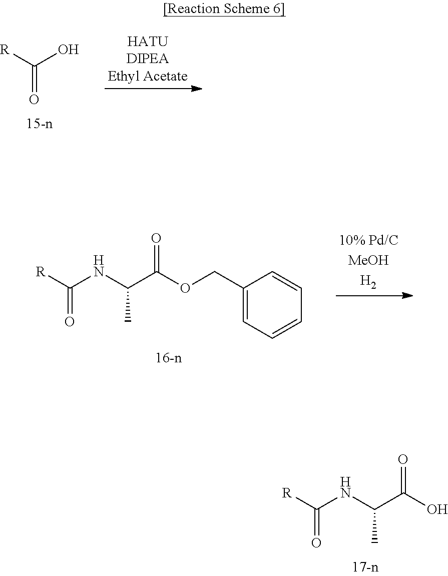

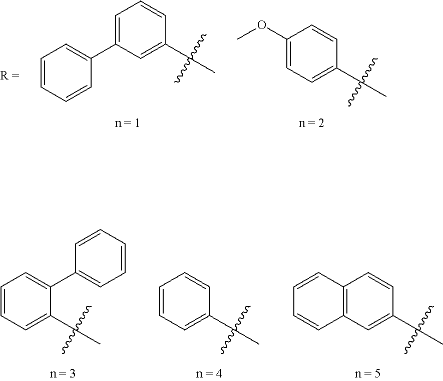

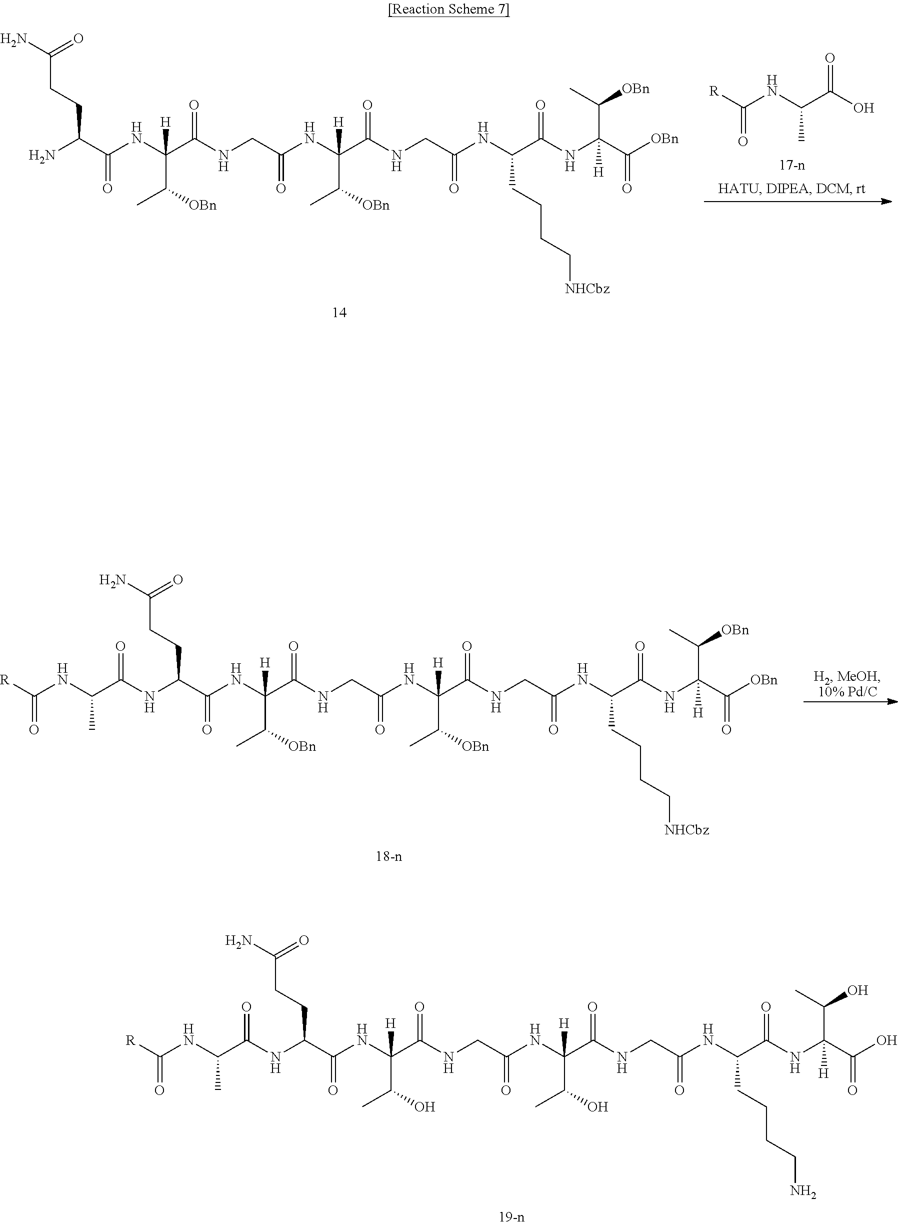

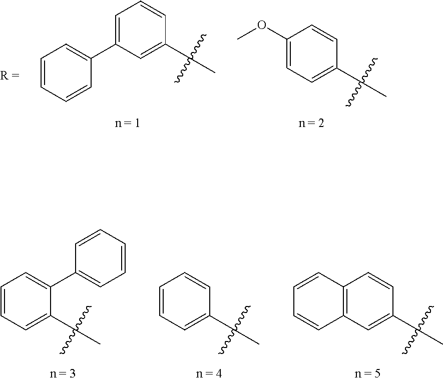

According to Reaction Scheme 7, 2.9 mg of 3-PhPh-AQTGTGKT (purity: 90% or more), 7.0 mg of 4-MeOPh-AQTGTGKT (purity: 89%), 22.7 mg of 2-PhPh-AQTGTGKT (purity: 95% or more), 23.2 mg of Ph-AQTGTGKT (purity: 90% or more), and 23.2 mg of naphthyl-AQTGTGKT (purity: 85%) were finally obtained.

Hereinafter, each analog synthesis process will be described in detail.

### 1.7.1. Synthesis of 3-PhPh-AQTGTGKT

The final target Compound 19-1 (3-PhPh-AQTGTGKT) was synthesized by reacting Compound 17-1 obtained according to Reaction Scheme 8 below with Compound 14 according to Reaction Scheme 9.

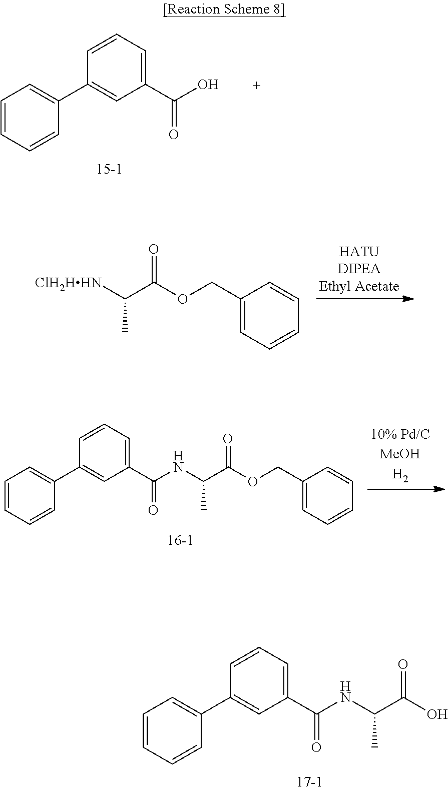

More specifically, H-Ala-OBzl·HCl (388 mg, 1.80 mmol, 1.2 eq) was suspended in ethyl acetate (10 mL), and N,N-diisopropylethylamine (653 μL, 3.75 mmol, 2.5 eq) was added. After stirring for 5 minutes at room temperature, HATU (855 mg, 2.25 mmol, 1.5 eq) and [1,1′-biphenyl]-3-carboxylic acid (297 mg, 1.50 mmol, 1 eq) were added, and the mixture was stirred for 2 hours at room temperature. The reaction mixture was diluted with ethyl acetate and then washed with NH₄Cl (saturated aqueous), NaHCO₃ (saturated aqueous), and brine. The obtained organic material was dehydrated (Na₂SO₄), filtered and concentrated under reduced pressure. The residue was purified on a 25 g column with a gradient of 2 to 40% ethyl acetate in heptane, thereby obtaining Compound 16-1 as a colorless solid (493 mg, 91% yield).

Subsequently, 10% Pd/C (49 mg) moistened with a minimal amount of water was added to a solution of Compound 16-1 (3-PhPh-AlaOBn; 493 mg, 1.37 mmol) in methanol (30 mL). The mixture was stirred for 2 hours under a hydrogen atmosphere (balloon). The mixture was filtered through a Celite pad and washed with methanol and ethyl acetate. The obtained filtrate was concentrated under reduced pressure to obtain Compound 17-1 as a colorless foam (337 mg, 91% yield), which was reacted with Compound 14 according to Reaction Scheme 9 below.

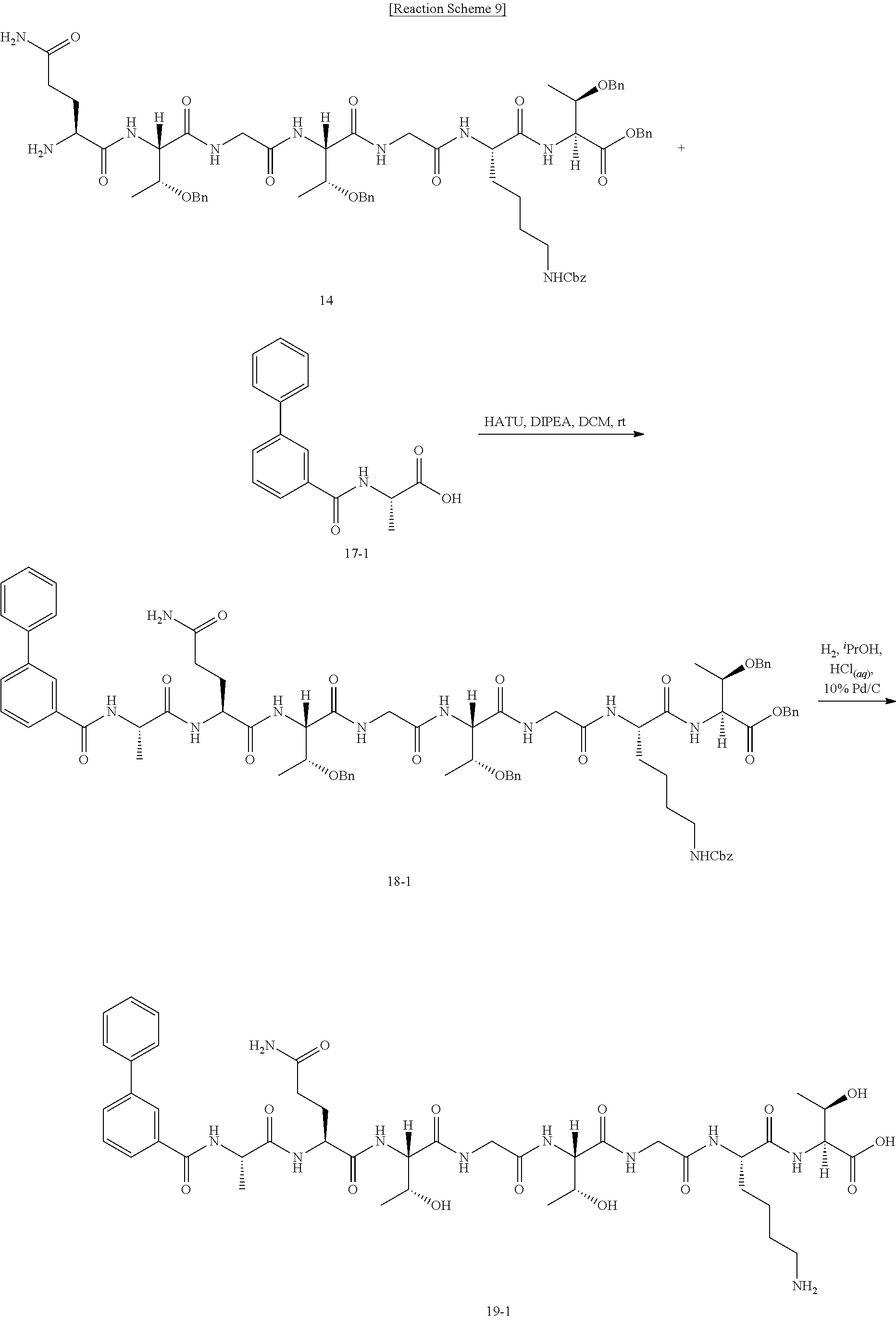

More specifically, HATU (106 mg, 0.278 mmol, 1.1 eq) was added to a suspension of GlnThr(OBn)GlyThr(OBn)GlyLys(Cbz) Thr(OBn)OBn (Compound 14; 300 mg, 0.253 mmol, 1 eq) and 3-PhPh-AlaOH (Compound 17-1; 68.0 mg, 0.253 mmol, 1 eq) in N, N-diisopropylethylamine (97.0 μL, 0.556 mmol, 2.2 eq) and dichloromethane (20 mL). The mixture was stirred for 2 hours at room temperature, and the reaction mixture was washed with NaHCO₃ (saturated aqueous). The organic material was concentrated under reduced pressure, and the residue was purified on a 60 g C18 column with a gradient of 40 to 100% acetonitrile (0.1% formic acid) in water (0.1% formic acid) and freeze-dried, thereby obtaining Compound 18-1 as a colorless solid (140 mg, 38% yield).

Subsequently, 10% Pd/C (85.0 mg) was added to a solution of 3-PhPh-AlaGlnThr(OBn)GlyThr(OBn)GlyLys(Cbz)Thr(OBn)OBn (Compound 18-1; 85.0 mg, 59.1 mol) in 2M hydrochloric acid (aqueous, 0.5 mL) and 2-propanol (10 mL). The mixture was stirred for 4.5 hours under a hydrogen atmosphere (balloon), and filtered through a 0.45 m syringe filter. The obtained filtrate was concentrated under reduced pressure, and the residue was freeze-dried. Afterward, the dried residue was purified on a 60 g C18 column using 5-50% acetonitrile (0.1% formic acid) in water (0.1% formic acid), and freeze-dried, thereby obtaining target Compound 19-1 as a colorless solid (2.9 mg, 5% yield).

The ¹H NMR data of Compound 19-1 (3-PhPh-AQTGTGKT) was measured as follows:

¹H NMR (400 MHz; D2O): δ=8.01-7.99 (m, 1H), 7.86-7.82 (m, 1H), 7.75-7.66 (m, 3H), 7.55 (t, J 7.7 Hz, 1H), 7.49 (t, J 7.5 Hz, 2H), 7.43-7.38 (m, 1H), 4.48-4.24 (m, 5H), 4.23-4.12 (m, 3H), 4.09 (d, J 3.8 Hz, 1H), 4.00-3.96 (m, 2H), 3.88 (s, 2H), 2.90 (t, J 7.4 Hz, 2H), 2.35 (t, J 7.5 Hz, 2H), 2.15-2.06 (m, 1H), 2.03-1.91 (m, 1H), 1.84-1.72 (m, 1H), 1.70-1.52 (m, 3H), 1.45 (d, J 7.2 Hz, 3H), 1.40-1.24 (m, 2H), 1.15-1.05 (m, 9H), 16 exchangeable protons not visible.

### 1.7.2. Synthesis of 4-MeOPh-AQTGTGKT

The final target Compound 19-2 (4-MeOPh-AQTGTGKT) was synthesized by reacting Compound 17-2 obtained according to Reaction Scheme 10 below with Compound 14 according to Reaction Scheme 11.

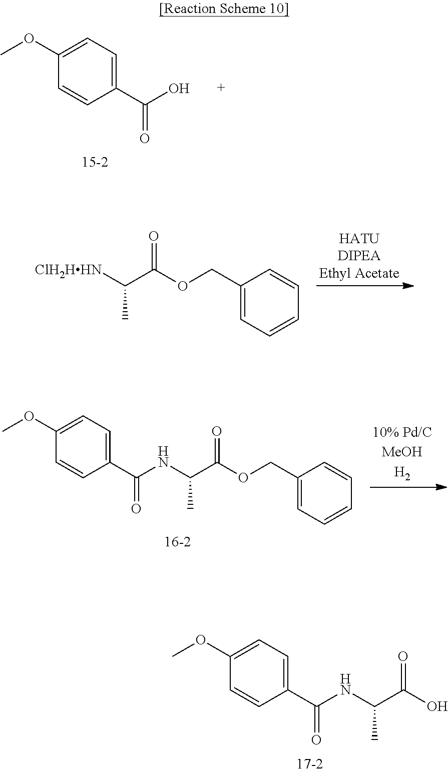

More specifically, H-Ala-OBzl·HCl (425 mg, 1.97 mmol, 1.2 eq) was suspended in ethyl acetate (10 mL), and N,N-diisopropylethylamine (715 μL, 4.11 mmol, 2.5 eq) was added. After stirred for 5 minutes at room temperature, HATU (937 mg, 2.46 mmol, 1.5 eq) and 4-methoxybenzoic acid (250 mg, 1.64 mmol, 1 eq) were added, and the mixture was stirred for 2 hours at room temperature. The reaction mixture was diluted with ethyl acetate and then washed with NH₄Cl (saturated aqueous), NaHCO₃ (saturated aqueous), and brine. The organic material was dehydrated (Na₂SO₄), filtered, and concentrated under reduced pressure. The residue was purified on a 25 g column with a gradient of 15 to 50% ethyl acetate in heptane, thereby obtaining Compound 16-2 as a colorless solid (360 mg, 70% yield).

Subsequently, 10% Pd/C (18 mg) moistened with a minimal amount of water was added to a solution of 4-OMePh-AlaOBn (Compound 16-2; 180 mg, 0.574 mmol) in methanol (15 mL). The mixture was stirred for 110 hours under a hydrogen atmosphere (balloon). Afterward, the mixture was filtered through a Celite pad and washed with methanol. The obtained filtrate was concentrated under reduced pressure and concentrated under reduced pressure, thereby obtaining Compound 17-2 as a colorless oil (128 mg, 100% yield), which was reacted with Compound 14 according to Reaction Scheme 11 below.

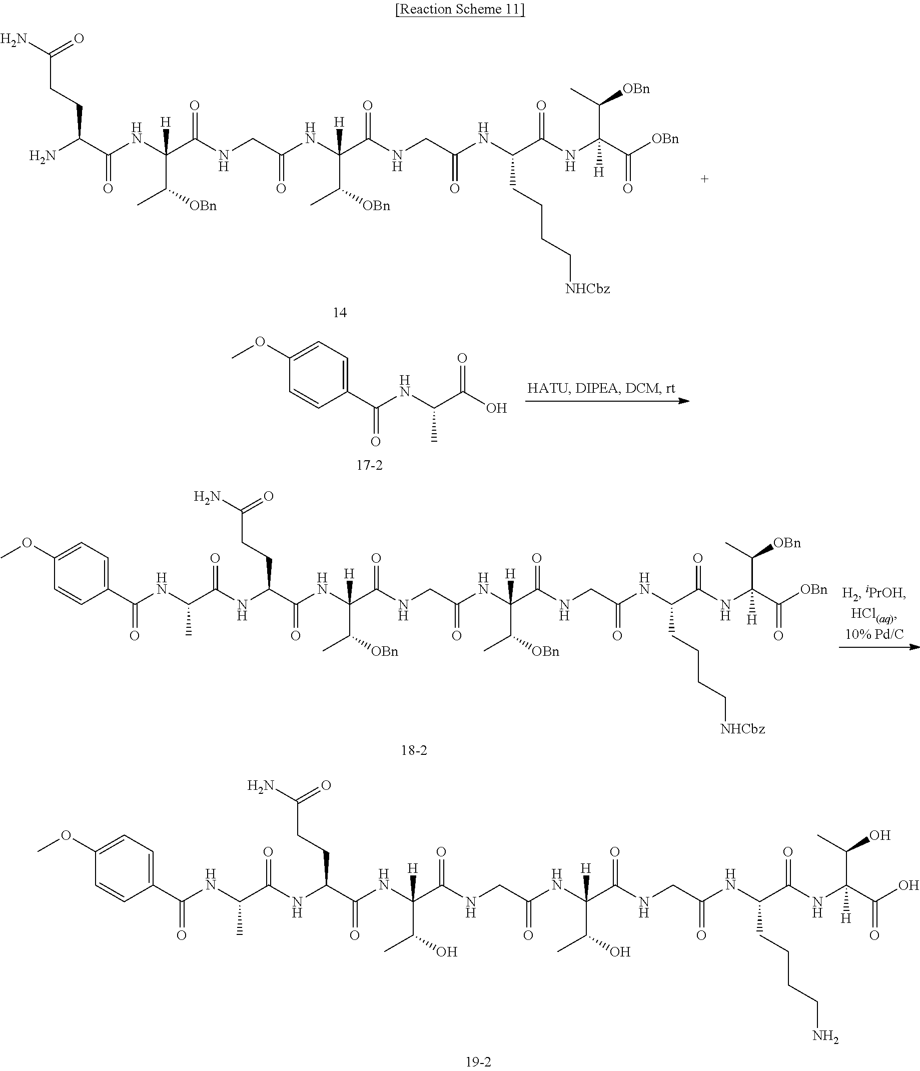

More specifically, HATU (74.5 mg, 0.196 mmol, 1.2 eq) was added to a suspension of GlnThr(OBn)GlyThr(OBn)GlyLys (Cbz)Thr(OBn)OBn (Compound 14; 200 mg, 0.164 mmol, 1 eq) and 4-OMePh-AlaOH (Compound 17-2; 36.6 mg, 0.164 mmol, 1 eq) in N, N-diisopropyl ethyl amine (63.0 μL, 0.360 mmol, 2.2 eq) and dichloromethane (20 mL). The mixture was stirred for 64 hours at room temperature, and the reaction mixture was diluted with methanol and then washed with NH₄Cl (saturated aqueous), NaHCO₃ (saturated aqueous), and water. The organic material was concentrated under reduced pressure and the residue was purified on a 60 g C18 column with a gradient of 50 to 95% acetonitrile (0.1% formic acid) in water (0.1% formic acid) and freeze-dried, thereby obtaining Compound 18-2 as a colorless solid (113 mg, 50% yield).

Subsequently, 10% Pd/C (100 mg) was added to a solution of 4-OMePh-AlaGlnThr(OBn)GlyThr(OBn)GlyLys(Cbz)Thr(OBn)OBn (Compound 18-2; 108 mg, 77.6 mol) in 2M hydrochloric acid (aqueous, 1.0 mL) and 2-propanol (20 mL). The mixture was stirred for 18 hours under a hydrogen atmosphere (balloon). The mixture was filtered through a 0.45 m syringe filter. The obtained filtrate was concentrated under reduced pressure, and the residue was dissolved in water and then freeze-dried. The dried residue was purified on a 30 g C18 column using a gradient of 5 to 30% acetonitrile (0.1% formic acid) in water (0.1% formic acid) and freeze-dried, thereby obtaining Compound 19-2 as a colorless solid (7.0 mg, 10% yield).

The ¹H NMR data of Compound 19-2 (4-MeOPh-AQTGTGKT) was measured as follows:

¹H NMR (400 MHz; D2O): δ=7.76-7.71 (m, 2H), 7.02-6.98 (m, 2H), 4.42-4.28 (m, 5H), 4.24-4.13 (m, 3H), 4.09 (d, J 3.9 Hz, 1H), 4.01-3.96 (m, 2H), 3.89 (s, 2H), 3.81 (s, 3H), 2.91 (t, J 7.4 Hz, 2H), 2.34 (t, J 7.6 Hz, 2H), 2.14-2.06 (m, 1H), 2.00-1.91 (m, 1H), 1.85-1.75 (m, 1H), 1.71-1.54 (m, 3H), 1.42 (d, J 7.2 Hz, 3H), 1.40-1.25 (m, 3H), 1.16-1.07 (m, 8H), 16 exchangeable protons not visible.

### 1.7.3. Synthesis of 2-PhPh-AQTGTGKT

The final target Compound 19-3 (2-PhPh-AQTGTGKT) was synthesized by reacting Compound 17-3 obtained according to Reaction Scheme 12 below with Compound 14 according to Reaction Scheme 13.

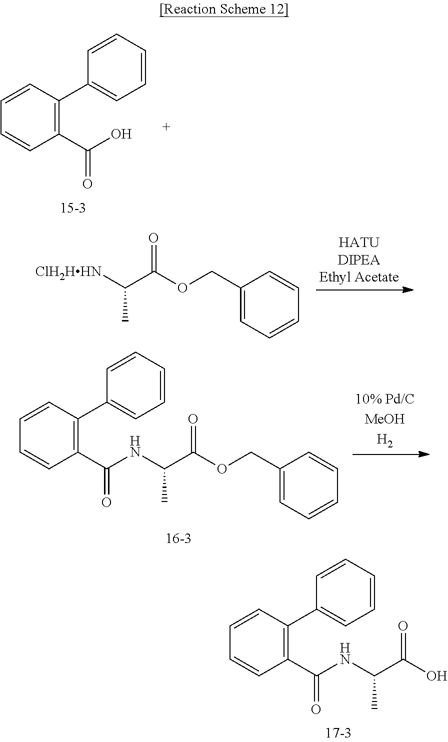

More specifically, H-Ala-OBzl·HCl (388 mg, 1.80 mmol, 1.2 eq) was suspended in ethyl acetate (10 mL), and N,N-diisopropylethylamine (653 μL, 3.75 mmol, 2.5 eq) was added. After stirring for 5 minutes at room temperature, HATU (855 mg, 2.25 mmol, 1.5 eq) and [1,1′-biphenyl]-3-carboxylic acid (297 mg, 1.50 mmol, 1 eq) were added, and the mixture was stirred for 2 hours at room temperature. The reaction mixture was diluted with ethyl acetate and then washed with NH₄Cl (saturated aqueous), NaHCO₃ (saturated aqueous), and brine. The obtained organic material was dehydrated (Na₂SO₄), filtered and concentrated under reduced pressure. The residue was purified on a 25 g column with a gradient of 2 to 40% ethyl acetate in heptane, thereby obtaining Compound 16-3 as a colorless oil (416 mg, 77% yield).

Subsequently, 10% Pd/C (42 mg) moistened with a minimal amount of water was added to a solution of 2-PhPh-AlaOBn (Compound 16-3; 416 mg, 1.16 mmol) in methanol (20 mL). The mixture was stirred for 18 hours under a hydrogen atmosphere (balloon). Afterward, the mixture was filtered through a Celite pad and washed with methanol. The obtained filtrate was concentrated under reduced pressure, thereby obtaining Compound 17-3 as a colorless oil (308 mg, 99% yield), which was reacted with Compound 14 according to Reaction Scheme 13 below.

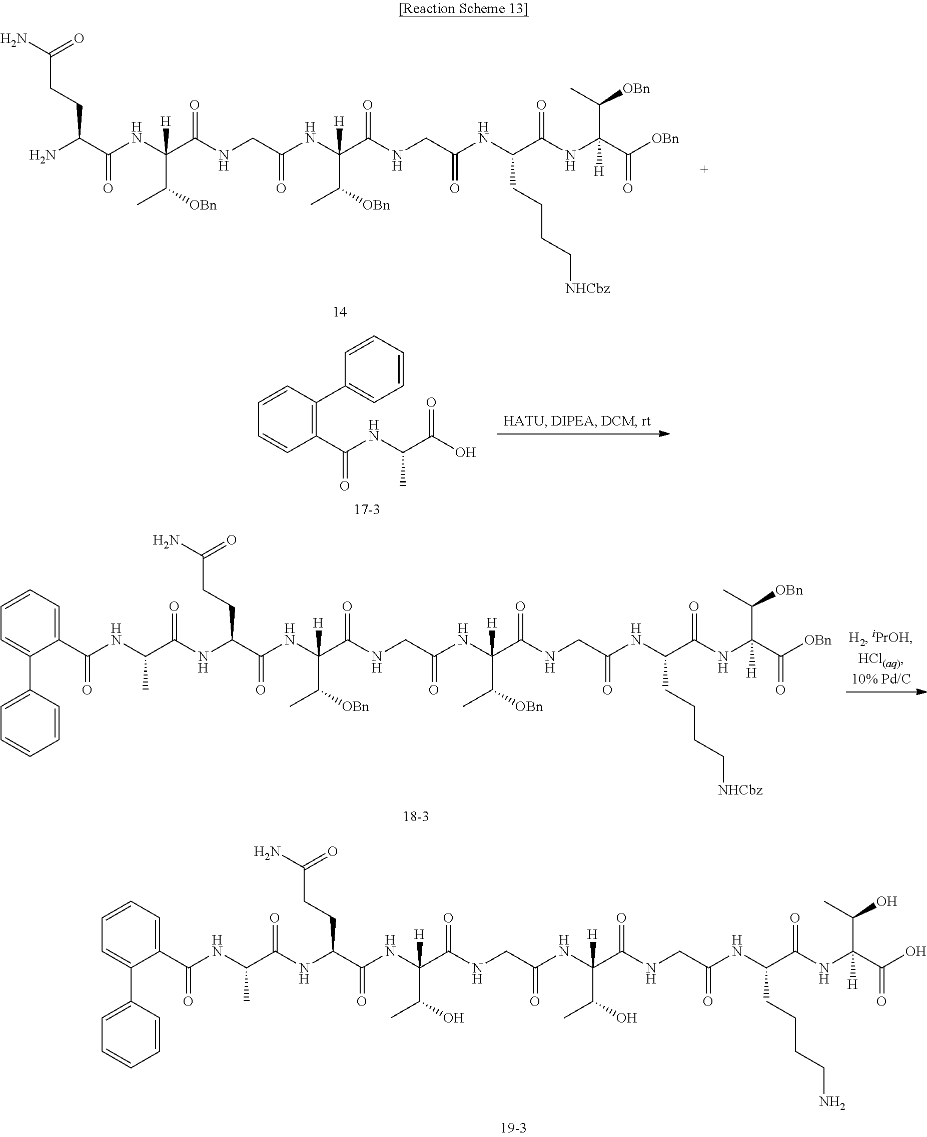

More specifically, HATU (74.5 mg, 0.196 mmol, 1.2 eq) was added to a suspension of GlnThr(OBn)GlyThr(OBn)GlyLys(Cbz) Thr(OBn)OBn (Compound 14; 200 mg, 0.164 mmol, 1 eq) and 2-PhPh-AlaOH (Compound 17-3; 44.2 mg, 0.164 mmol, 1 eq) in N,N-diisopropyl ethyl amine (63.0 μL, 0.360 mmol, 2.2 eq) and dichloromethane (20 mL). The mixture was stirred for 64 hours at room temperature, and the reaction mixture was diluted with methanol and then washed with NH₄Cl (saturated aqueous), NaHCO₃ (saturated aqueous), and water. The organic layer was concentrated under reduced pressure, and the residue was purified on a 60 g C18 column with a gradient of 50 to 95% acetonitrile (0.1% formic acid) in water (0.1% formic acid) and freeze-dried, thereby obtaining Compound 18-3 as a colorless solid (115 mg, 49% yield).

Subsequently, 10% Pd/C (100 mg) was added to a solution of 2-PhPh-AlaGlnThr(OBn)GlyThr(OBn)GlyLys(Cbz)Thr(OBn)OBn (Compound 18-3; 110 mg, 76.5 mol) in 2M hydrochloric acid (aqueous, 1.0 mL) and 2-propanol (20 mL). The mixture was stirred for 18 hours under a hydrogen atmosphere (balloon). The mixture was filtered through a 0.45 m syringe filter. The obtained filtrate was concentrated under reduced pressure, and the residue was dissolved in water and then freeze-dried. The dried residue was purified on a 30 g C18 column with a gradient of 5 to 50% acetonitrile (0.1% formic acid) in water (0.1% formic acid) and freeze-dried, thereby obtaining Compound 19-3 as a colorless solid (22.7 mg, 31% yield).

The ¹H NMR data of Compound 19-3 (2-PhPh-AQTGTGKT) was measured as follows:

¹H NMR (400 MHz; D2O): δ=7.56-7.48 (m, 2H), 7.44-7.33 (m, 7H), 4.38-4.26 (m, 4H), 4.25-4.13 (m, 4H), 4.08 (d, J 3.9 Hz, 1H), 4.00-3.96 (m, 2H), 3.89 (s, 2H), 2.91 (t, J 7.5 Hz, 2H), 2.25 (t, J 7.5 Hz, 2H), 2.09-2.01 (m, 1H), 1.93-1.77 (m, 2H), 1.72-1.56 (m, 3H), 1.41-1.29 (m, 2H), 1.18 (d, J 7.2 Hz, 3H), 1.12 (d, J 5.6 Hz, 6H), 1.08 (d, J 6.4 Hz, 3H), 16 exchangeable protons not visible.

### 1.7.4. Synthesis of Ph-AQTGTGKT

The final target Compound 19-4 (Ph-AQTGTGKT) was synthesized by reacting Compound 17-4 obtained according to Reaction Scheme 14 below with Compound 14 according to Reaction Scheme 15.

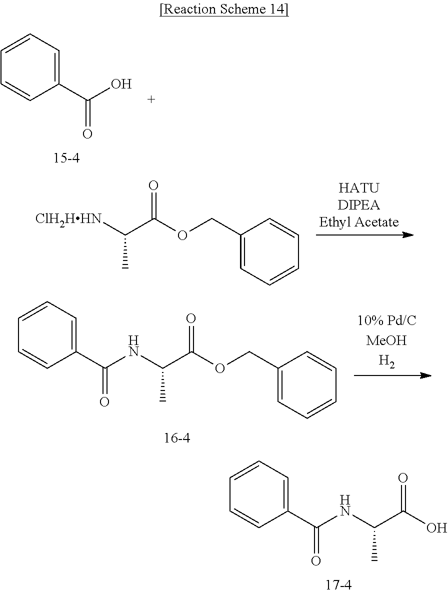

More specifically, H-Ala-OBzl·HCl (388 mg, 1.80 mmol, 1.2 eq) was suspended in ethyl acetate (10 mL), and N,N-diisopropylethylamine (653 μL, 3.75 mmol, 2.5 eq) was added. After stirring for 5 minutes at room temperature, HATU (855 mg, 2.25 mmol, 1.5 eq) and benzoic acid (183 mg, 1.50 mmol, 1 eq) were added, and the mixture was stirred for 17 hours at room temperature. The reaction mixture was diluted with ethyl acetate and then washed with NH₄Cl (saturated aqueous), NaHCO₃ (saturated aqueous), and brine. The obtained organic material was dehydrated (Na₂SO₄), filtered and concentrated under reduced pressure. The residue was purified on a 25 g column with a gradient of 2 to 50% ethyl acetate in heptane, thereby obtaining Compound 16-4 as a colorless oil (375 mg, 88% yield).

Subsequently, 10% Pd/C (38 mg) moistened with a minimal amount of water was added to a solution of Ph-Ala-OBn (Compound 16-4; 375 mg, 1.32 mmol) in methanol (30 mL). The mixture was stirred for 4 hours under a hydrogen atmosphere (balloon). Afterward, the mixture was filtered through a Celite pad and washed with methanol. The obtained filtrate was concentrated under reduced pressure, thereby obtaining Compound 17-4 as colorless glass (glass; 254 mg, 99% yield), which was reacted with Compound 14 according to Reaction Scheme 15 as follows.

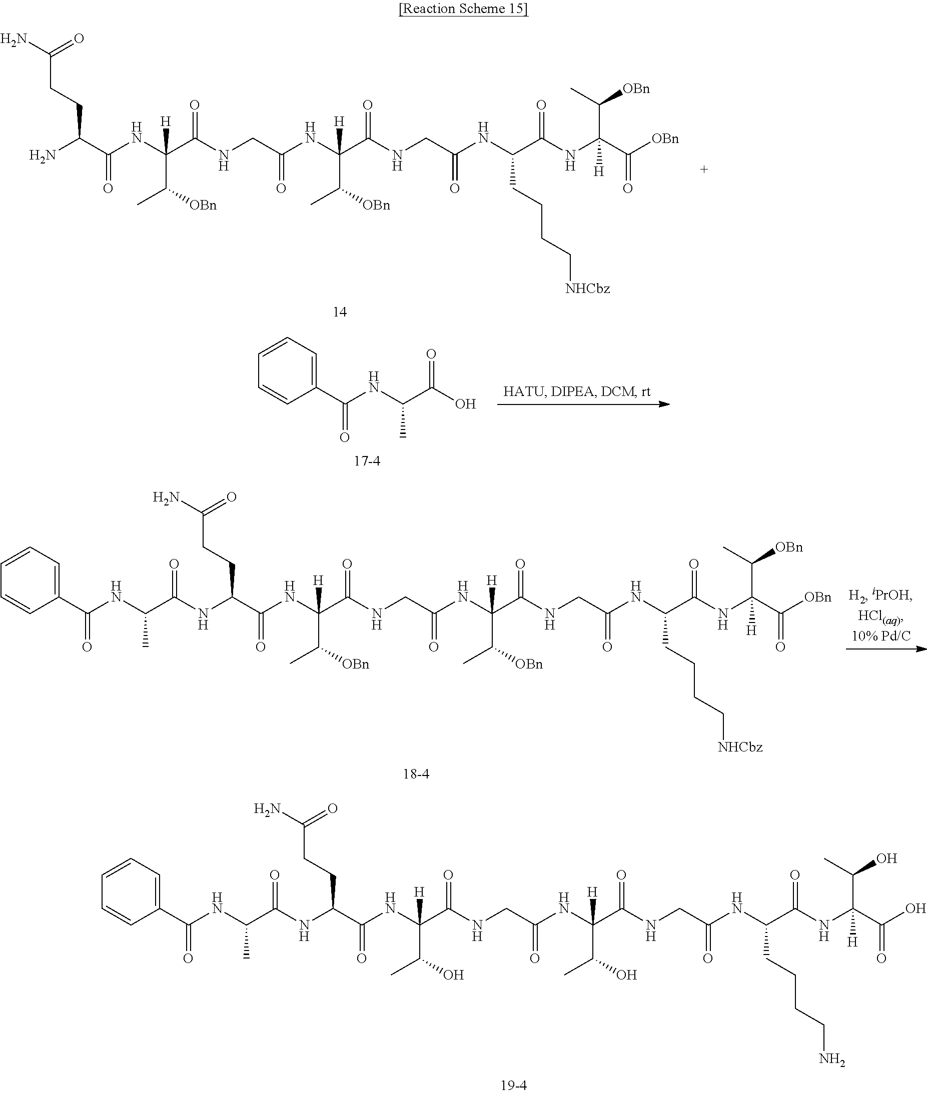

More specifically, HATU (106 mg, 0.278 mmol, 1.1 eq) was added to a suspension of GlnThr(OBn)GlyThr(OBn)GlyLys(Cbz)Thr (OBn)OBn (Compound 14; 300 mg, 0.253 mmol, 1 eq) and Ph-Ala-OH (Compound 17-4; 49.0 mg, 0.253 mmol, 1) in N,N-diisopropylethylamine (97.0 μL, 0.556 mmol, 2.2 eq) and dichloromethane (20 mL). The mixture was stirred for 2 hours at room temperature, and the reaction mixture was washed with NaHCO₃ (saturated aqueous). The organic material was concentrated under reduced pressure, and the obtained residue was purified on a 60 g C18 column with a gradient of 40 to 100% acetonitrile (0.1% formic acid) in water (0.1% formic acid) and freeze-dried, thereby obtaining Compound 18-4 as a colorless solid (200 mg, 58%).

Subsequently, 10% Pd/C (110 mg) was added to a solution of Ph-AlaGlnThr(OBn)GlyThr(OBn)GlyLys(Cbz)Thr(OBn)OBn (Compound 18-4; 110 mg, 80.8 mol) in 2M hydrochloric acid (aqueous, 1.0 mL) and 2-propanol (20 mL). The mixture was stirred for 18 hours under a hydrogen atmosphere (balloon). The mixture was filtered through a 0.45 m syringe filter. The obtained filtrate was concentrated under reduced pressure, and the residue was dissolved in water and then freeze-dried. The dried residue was purified on a 60 g C18 column using a gradient of 5 to 50% acetonitrile (0.1% formic acid) in water (0.1% formic acid) and freeze-dried, thereby obtaining Compound 19-4 as a colorless solid (23.2 mg, 33% yield).

The ¹H NMR data of Compound 19-4 (Ph-AQTGTGKT) was measured as follows:

¹H NMR (400 MHz; D2O): δ=7.74-7.70 (m, 2H), 7.58-7.53 (m, 1H), 7.48-7.43 (m, 2H), 4.45-4.34 (m, 3H), 4.32-4.27 (m, 2H), 4.25-4.14 (m, 3H), 4.11 (d, J 3.8 Hz, 1H), 4.00-3.96 (m, 2H), 3.89 (s, 2H), 2.91 (t, J 7.4 Hz, 2H), 2.34 (t, J 7.6 Hz, 2H), 2.14-2.06 (m, 1H), 2.01-1.91 (m, 1H), 1.85-1.76 (m, 1H), 1.71-1.56 (m, 3H), 1.43 (d, J 7.3 Hz, 3H), 1.40-1.30 (m, 2H), 1.16-1.07 (m, 9H), 16 exchangeable protons not visible.

### 1.7.5. Synthesis of Naphthyl-AQTGTGKT

The final target Compound 19-5 (naphthyl-AQTGTGKT) was synthesized by reacting Compound 17-5 obtained according to Reaction Scheme 16 with Compound 14 according to Reaction Scheme 17.

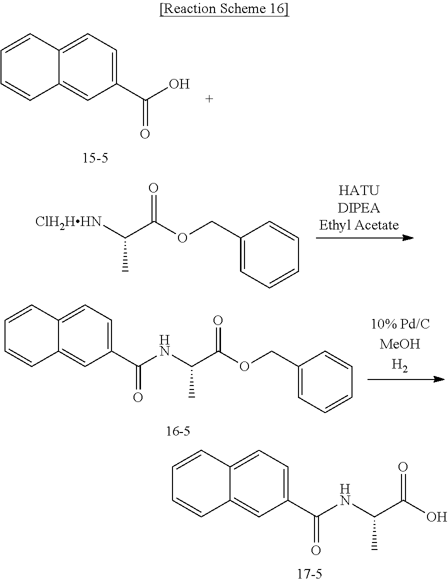

More specifically, H-Ala-OBzl·HCl (388 mg, 1.80 mmol, 1.2 eq) was suspended in ethyl acetate (10 mL), and N,N-diisopropylethylamine (653 μL, 3.75 mmol, 2.5 eq) was added. After stirring for 5 minutes at room temperature, HATU (855 mg, 2.25 mmol, 1.5 eq) and 2-naphthoic acid (258 mg, 1.50 mmol, 1 eq) were added, and then the mixture was stirred for 2 hours at room temperature. The reaction mixture was diluted with ethyl acetate and then washed with NH₄Cl (saturated aqueous), NaHCO₃ (saturated aqueous), and brine. The obtained organic material was dehydrated (Na₂SO₄), filtered, and concentrated under reduced pressure. The residue was purified on a 25 g column with a gradient of 2 to 40% ethyl acetate in heptane, thereby obtaining Compound 16-5 as a colorless solid (385 mg, 77% yield).

Subsequently, 10% Pd/C (39 mg) moistened with a minimal amount of water was added to a solution of 2-Naphthyl-AlaOBn (Compound 16-5; 385 mg, 1.15 mmol) in methanol (20 mL). The mixture was stirred for 4 hours under a hydrogen atmosphere (balloon). Afterward, the mixture was filtered through a Celite pad and washed with methanol. The obtained filtrate was concentrated under reduced pressure, thereby obtaining Compound 17-5 as a colorless glass (266 mg, 95% yield), which was reacted with Compound 14 according to Reaction Scheme 17 below.

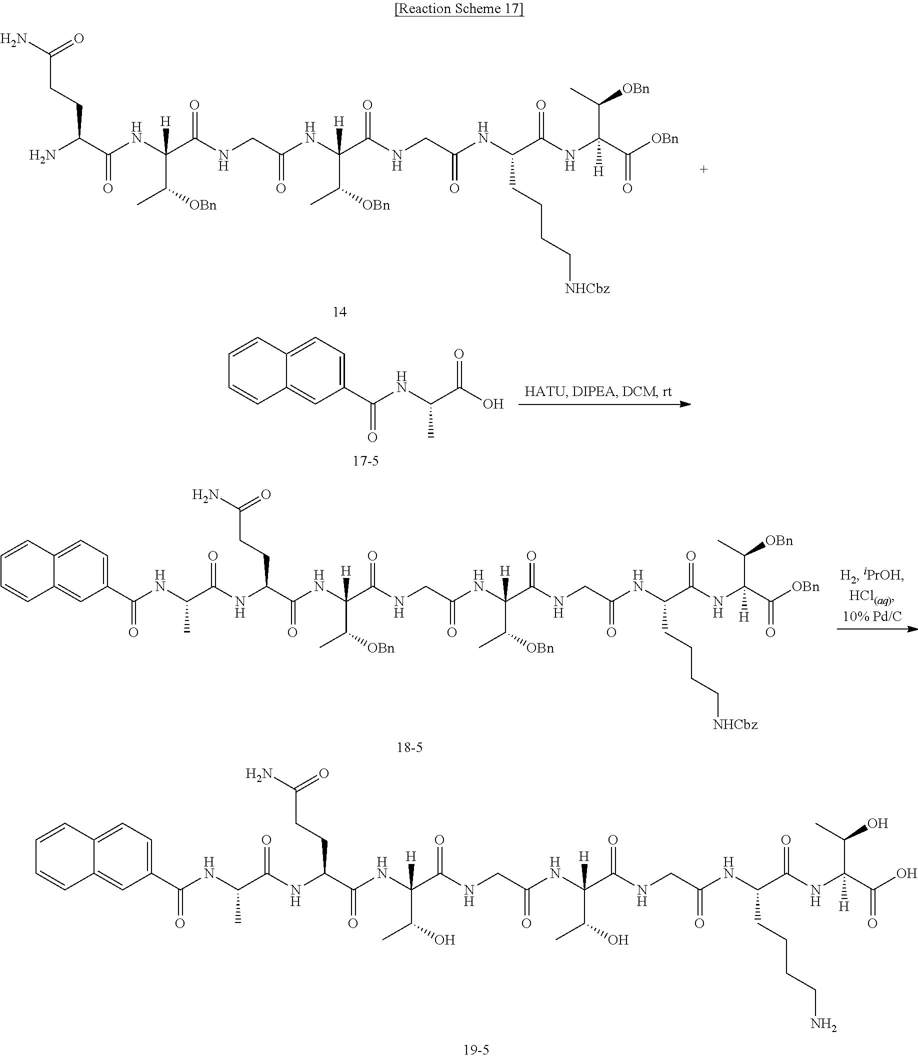

More specifically, HATU (106 mg, 0.278 mmol, 1.1 eq) was added to a suspension of GlnThr(OBn)GlyThr(OBn)GlyLys(Cbz) Thr(OBn)OBn (Compound 14; 300 mg, 0.253 mmol, 1 eq) and 2-Naphthyl-Ala-OH (Compound 17-5; 61.0 mg, 0.253 mmol, 1 eq) in N,N-diisopropylethylamine (97.0 μL, 0.556 mmol, 2.2 eq) and dichloromethane (20 mL). The mixture was stirred for 2 hours at room temperature and then washed with (saturated aqueous) NaHCO₃. The obtained organic layer was concentrated under reduced pressure, and the residue was purified on a 60 g C18 column with a gradient of 40 to 100% acetonitrile (0.1% formic acid) in water (0.1% formic acid) and freeze-dried, thereby obtaining Compound 18-5 as a colorless solid (230 mg, 64% yield).

Subsequently, 10% Pd/C (101 mg) was added to a solution of 2-Naphthyl-AlaGlnThr(OBn)GlyThr(OBn)GlyLys(Cbz)Thr(OBn)OBn (Compound 18-5; 101 mg, 71.5 mol) in 2M hydrochloric acid (aqueous, 1.0 mL) and 2-propanol (20 mL). The mixture was stirred for 3 hours under a hydrogen atmosphere (balloon). The mixture was filtered through a 0.45 m syringe filter. The obtained filtrate was concentrated under reduced pressure, and the residue was dissolved in water and then freeze-dried. The dried residue was purified on a 30 g C18 column with a gradient of 5 to 40% acetonitrile (0.1% formic acid) in water (0.1% formic acid) and freeze-dried, thereby obtaining Compound 19-5 as a colorless solid (23.2 mg, 33% yield).

The ¹H NMR data of Compound 19-5 (Naphthyl-AQTGTGKT) was measured as follows:

¹H NMR (400 MHz; D2O): δ=8.32 (s, 1H), 8.01-7.90 (m, 3H), 7.79-7.73 (m, 1H), 7.64-7.55 (m, 2H), 4.51-3.70 (m, 13H), 2.96-2.81 (m, 2H), 2.39-2.30 (m, 2H), 2.18-2.04 (m, 1H), 2.03-1.93 (m, 1H), 1.85-1.72 (m, 1H), 1.71-1.53 (m, 3H), 1.50-1.43 (m, 3H), 1.39-1.24 (m, 2H), 1.19-1.04 (m, 9H), 16 exchangeable protons not visible.

### 1.8. Synthesis of Ac-AQTGTGKT

The final target Compound 19-6 (Ac-AQTGTGKT) was synthesized according to Reaction Scheme 18 below.

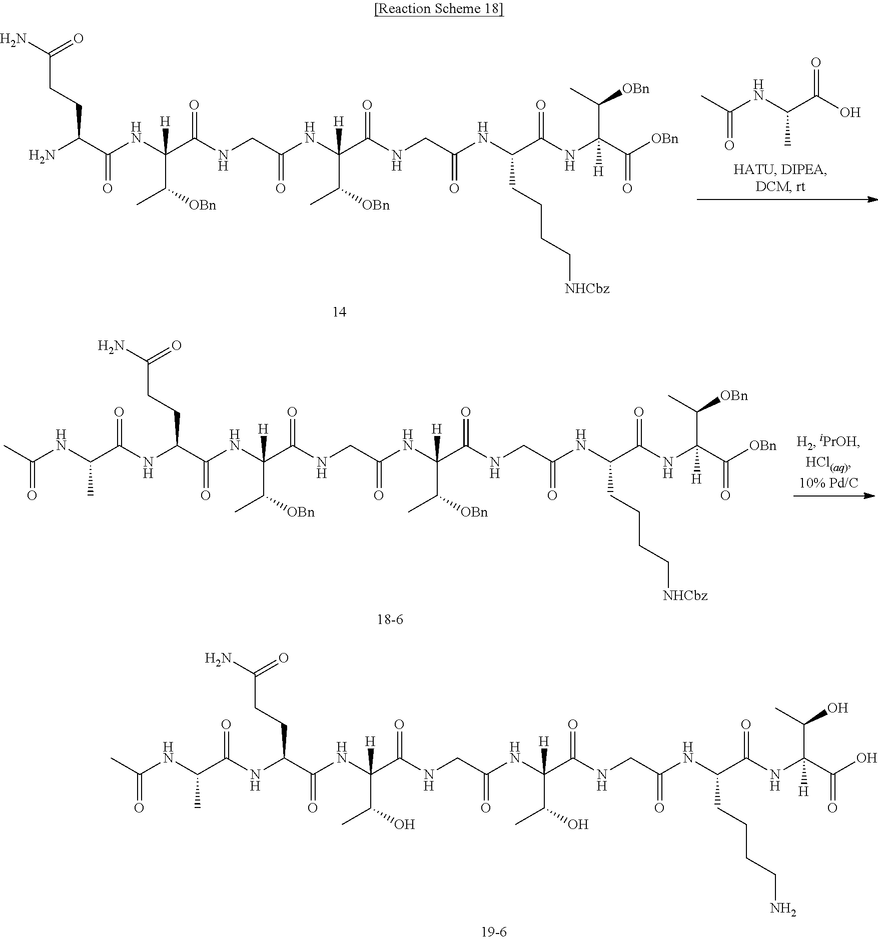

More specifically, HATU (112 mg, 0.294 mmol, 1.2 eq) was added to a suspension of GlnThr(OBn)GlyThr(OBn)GlyLys(Cbz) Thr(OBn)OBn (Compound 14; 300 mg, 0.245 mmol, 1 eq) and Ac-Ala-OH (32.1 mg, 0.245 mmol, 1 eq) in N, N-diisopropylethylamine (94.0 μL, 0.540 mmol, 2.2 eq) and dichloromethane (30 mL). The mixture was stirred for 14 hours at room temperature, and the reaction mixture was washed with (saturated aqueous) NH₄Cl, NaHCO₃, and water. The organic layer was concentrated under reduced pressure, and the residue was purified on a 60 g C18 column with a gradient of 50 to 95% acetonitrile (0.1% formic acid) in water (0.1% formic acid). The desired fractions were combined and freeze-dried, thereby obtaining Compound 18-6 as a colorless solid (104 mg, 33% yield).

Subsequently, 10% Pd/C (10 mg) was added to a solution of Ac-AlaGlnThr(OBn)GlyThr(OBn)GlyLys(Cbz)Thr(OBn)OBn (Compound 18-6; 33 mg, 25 mol) in 2M hydrochloric acid (aqueous, 0.19 mL) and 2-propanol (5 mL). The mixture was stirred for 14 hours under a hydrogen atmosphere (balloon). The mixture was filtered through a 0.45 m syringe filter. The obtained filtrate was concentrated under reduced pressure, dissolved in water, and freeze-dried. The dried material was purified in a 500 mg SCX-2 cartridge while being eluted with 0.5M ammonia. The desired fractions were combined, concentrated under reduced pressure, and freeze-dried, thereby obtaining Compound 19-6 as a colorless solid (7.6 mg, 37% yield).

The ¹H NMR data of Compound 19-6 (Ac-AQTGTGKT) was measured as follows:

¹H NMR (400 MHz; D2O): δ=4.40-4.33 (m, 2H), 4.32-4.27 (m, 2H), 4.24-4.12 (m, 4H), 4.08 (d, J 4.0 Hz, 1H), 4.03-3.88 (m, 4H), 2.92 (t, J 7.2 Hz, 2H), 2.32 (t, J 7.8 Hz, 2H), 2.15-2.02 (m, 1H), 1.99-1.88 (m, 4H), 1.86-1.76 (m, 1H), 1.74-1.55 (m, 3H), 1.45-1.31 (m, 2H), 1.29 (t, J 7.4 Hz, 2H), 1.20-1.06 (m, 10H), 16 exchangeable protons not visible.

### 1.9. Synthesis of 4-PhPh-AQTGTGKT

### 1.9.1. Synthesis of QTGTGKT Intermediate

An intermediate QTGTGKT for 4-PhPh-AQTGTGKT synthesis was synthesized according to Reaction Schemes 19 to 23 below:

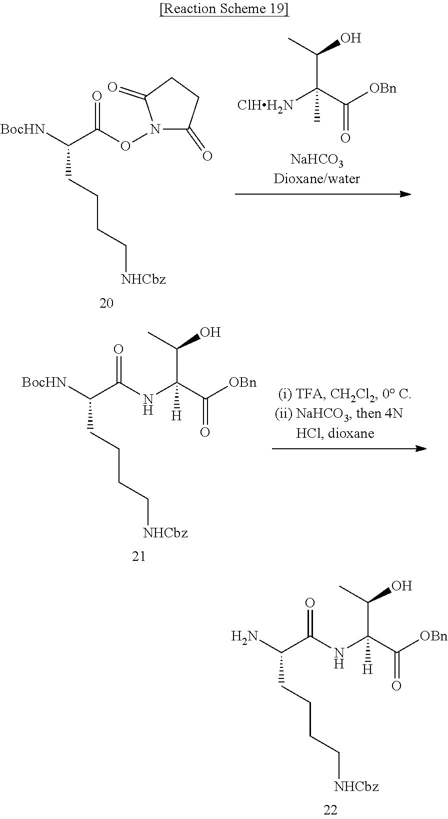

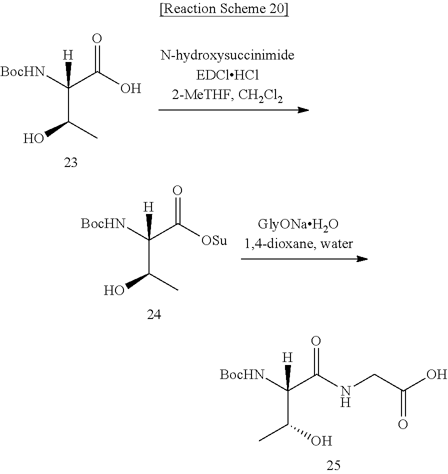

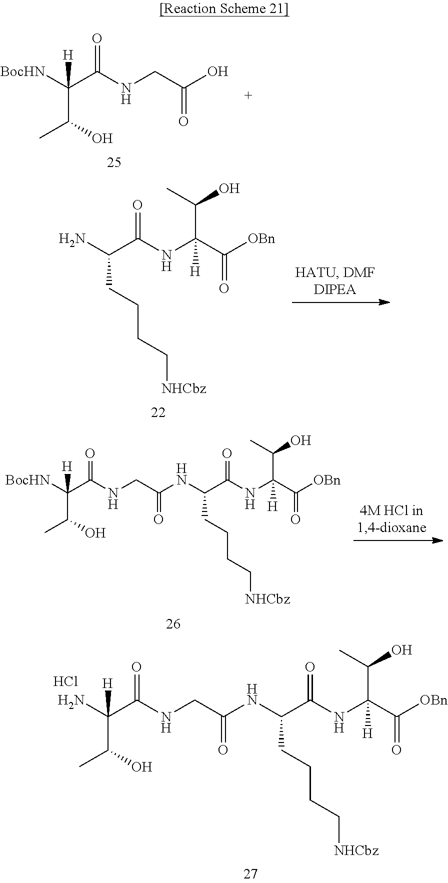 [SMILES: C[C@@H](O)[C@H](NC(=O)OC(C)(C)C)C(=O)NCC(=O)O ; C[C@@H](O)[C@H](NC(=O)[C@@H](N)CCCCNC(=O)OCc1ccccc1)C(=O)OCc1ccccc1 ; C[C@@H](O)[C@H](NC(=O)OC(C)(C)C)C(=O)NCC(=O)N[C@@H](CCCCNC(=O)OCc1ccccc1)C(=O)N[C@H](C(=O)OCc1ccccc1)[C@@H](C)O ; Cl ; C[C@@H](O)[C@H](N)C(=O)NCC(=O)N[C@@H](CCCCNC(=O)OCc1ccccc1)C(=O)N[C@H](C(=O)OCc1ccccc1)[C@@H](C)O]

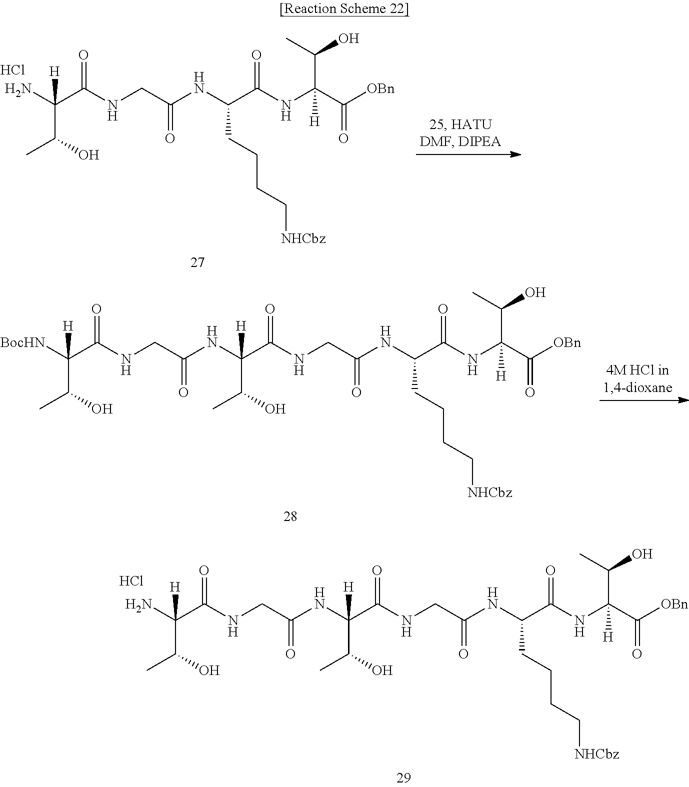 [SMILES: Cl ; C[C@@H](O)[C@H](N)C(=O)NCC(=O)N[C@@H](CCCCNC(=O)OCc1ccccc1)C(=O)N[C@H](C(=O)OCc1ccccc1)[C@@H](C)O ; C[C@@H](O)[C@H](NC(=O)CNC(=O)[C@@H](NC(=O)OC(C)(C)C)[C@@H](C)O)C(=O)NCC(=O)N[C@@H](CCCCNC(=O)OCc1ccccc1)C(=O)N[C@H](C(=O)OCc1ccccc1)[C@@H](C)O ; Cl ; C[C@@H](O)[C@H](N)C(=O)NCC(=O)N[C@H](C(=O)NCC(=O)N[C@@H](CCCCNC(=O)OCc1ccccc1)C(=O)N[C@H](C(=O)OCc1ccccc1)[C@@H](C)O)[C@@H](C)O]

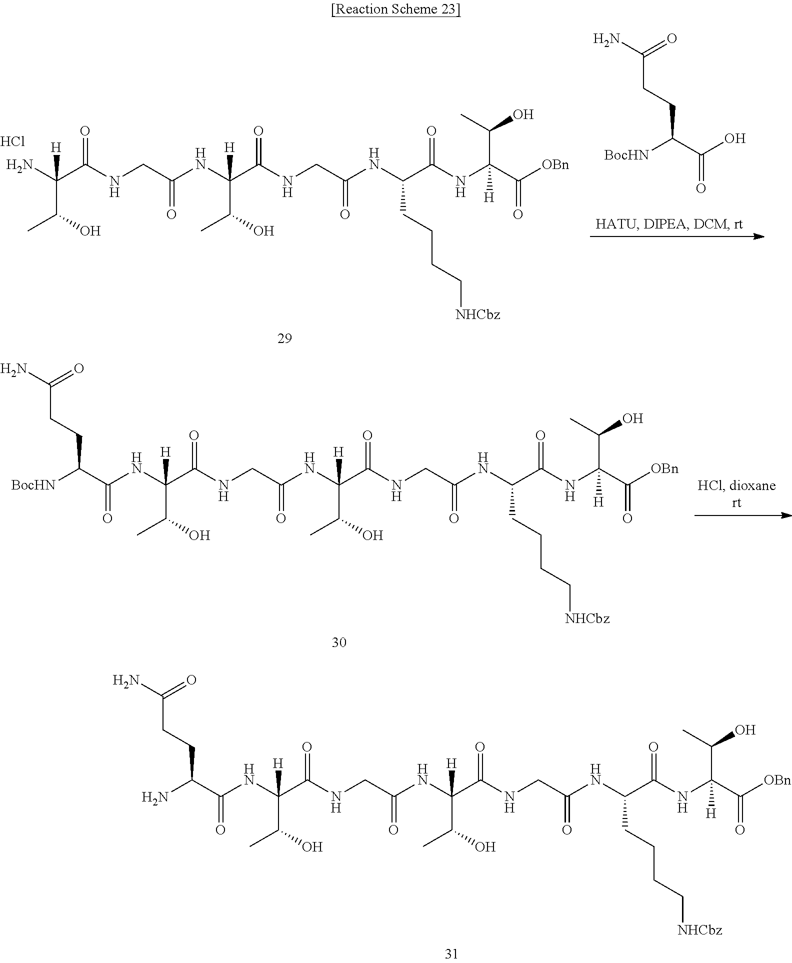 [SMILES: Cl ; C[C@@H](O)[C@H](N)C(=O)NCC(=O)N[C@H](C(=O)NCC(=O)N[C@@H](CCCCNC(=O)OCc1ccccc1)C(=O)N[C@H](C(=O)OCc1ccccc1)[C@@H](C)O)[C@@H](C)O ; CC(C)(C)OC(=O)N[C@@H](CCC(N)=O)C(=O)O ; C[C@@H](O)[C@H](NC(=O)CNC(=O)[C@@H](NC(=O)[C@H](CCC(N)=O)NC(=O)OC(C)(C)C)[C@@H](C)O)C(=O)NCC(=O)N[C@@H](CCCCNC(=O)OCc1ccccc1)C(=O)N[C@H](C(=O)OCc1ccccc1)[C@@H](C)O ; C[C@@H](O)[C@H](NC(=O)CNC(=O)[C@@H](NC(=O)[C@@H](N)CCC(N)=O)[C@@H](C)O)C(=O)NCC(=O)N[C@@H](CCCCNC(=O)OCc1ccccc1)C(=O)N[C@H](C(=O)OCc1ccccc1)[C@@H](C)O]

Reaction Schemes 19 to 23 are almost similar processes to Reaction Schemes 1 to 5, except for the presence or absence of a benzyl protective group, and thus overlapping description will be omitted.

### 1.9.2. Synthesis of 4-PhPh-AQTGTGKT

The final product 4-PhPh-AQTGTGKT was obtained by combining Compound 31 obtained in Reaction Scheme 23 with an alanine derivative synthesized in Reaction Scheme 24 below according to Reaction Scheme 25.

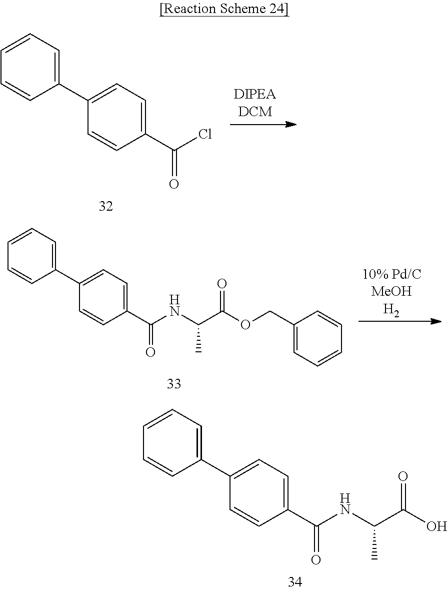 [SMILES: O=C(Cl)c1ccc(-c2ccccc2)cc1 ; C[C@H](NC(=O)c1ccc(-c2ccccc2)cc1)C(=O)OCc1ccccc1 ; C[C@H](NC(=O)c1ccc(-c2ccccc2)cc1)C(=O)O]

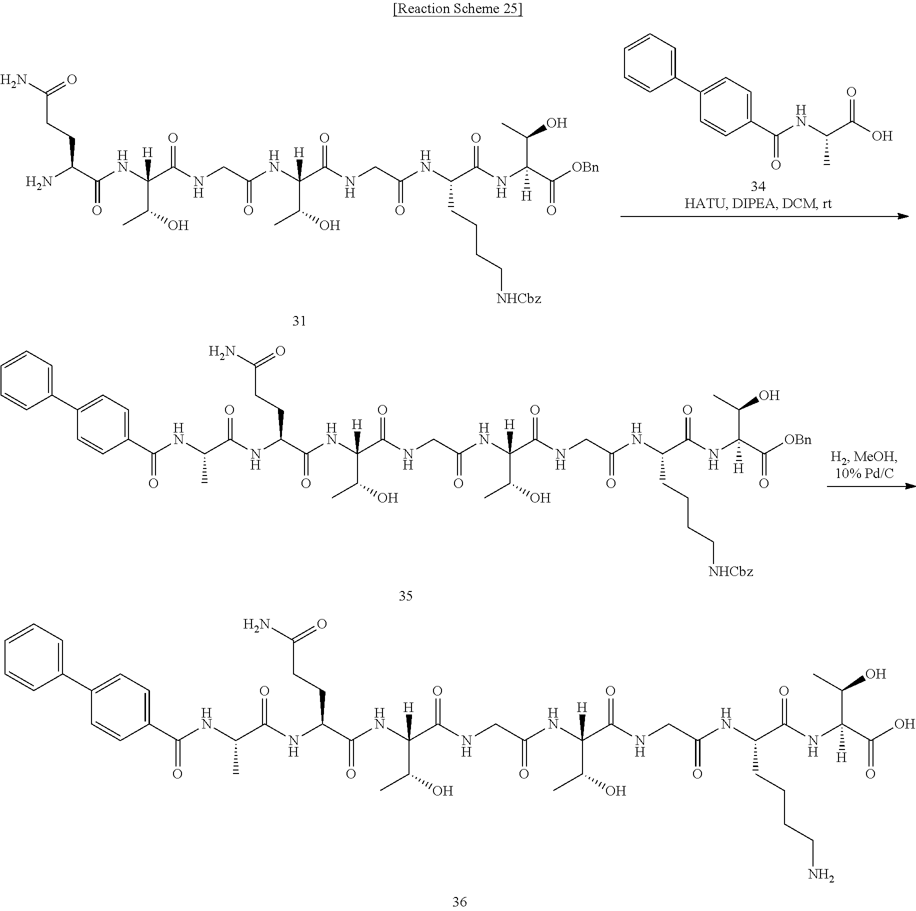

Reaction Schemes 24 and 25 were also carried out in an almost similar manner to the above-described Reaction Scheme, and overlapping description will be omitted. Finally, Compound 36 as a colorless solid (583 mg, 68% yield) was obtained.

The ¹H NMR data of Compound 36 (4-PhPh-AQTGTGKT) was measured as follows:

¹H NMR (400 MHz; D2O): δ=7.85 (d, J 8.8 Hz, 2H), 7.77 (d, J 8.8 Hz, 2H), 7.70 (d, J 7.2 Hz, 2H), 7.49 (t, J 7.2 Hz, 2H), 7.42 (t, J 7.1 Hz, 1H), 4.34-4.04 (m, 6H), 4.07 (d, J 3.6 Hz, 1H), 3.93 (d, J 2.0 Hz, 2H), 2.82 (t, J 7.0 Hz, 2H), 1.82-1.67 (m, 1H), 1.66-1.55 (m, 1H), 1.54-1.44 (m, 2H), 1.37-1.22 (m, 2H), 1.18 (d, J 6.4 Hz, 3H), 1.06 (t, J 6.5 Hz, 3H), 10 exchangeable protons not visible.

### 1.10. Identification of Compounds

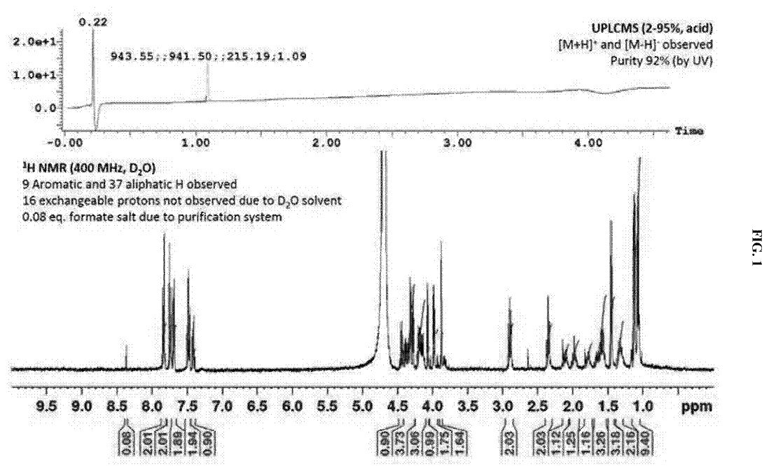

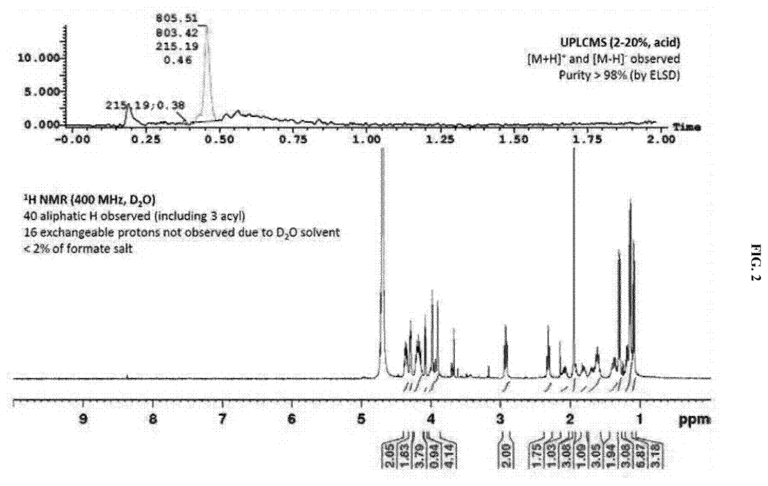

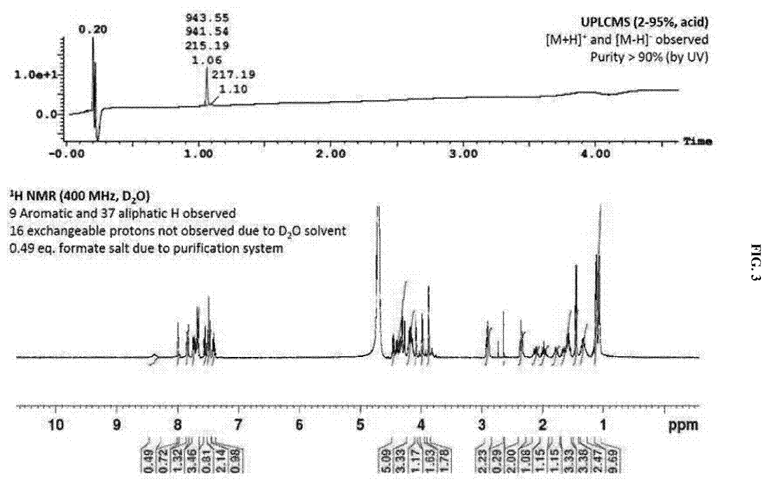

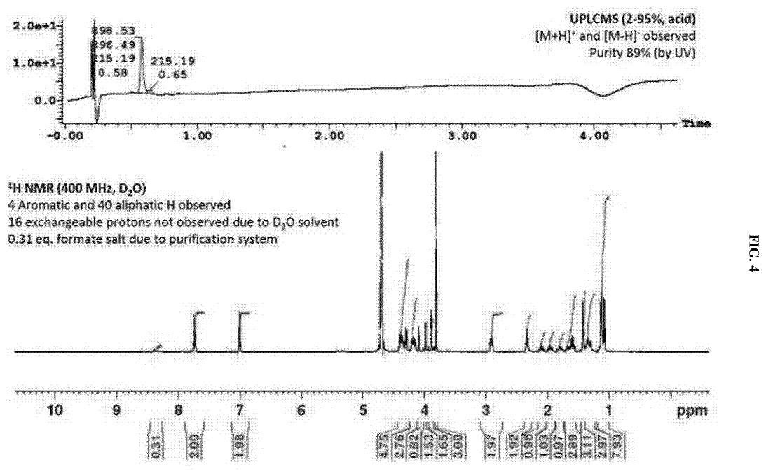

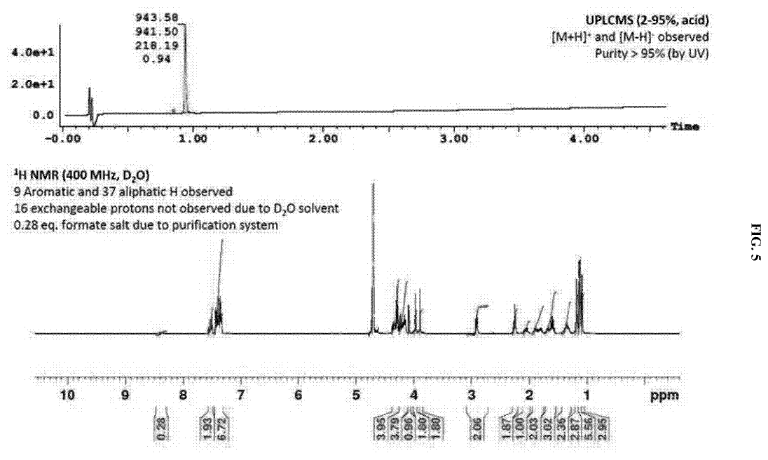

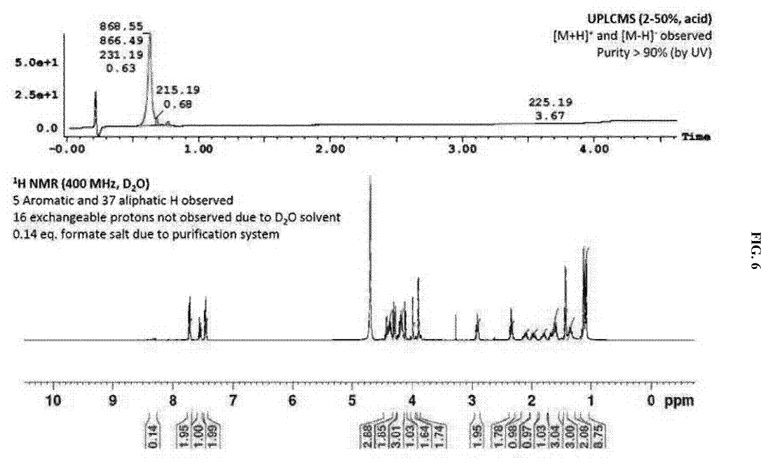

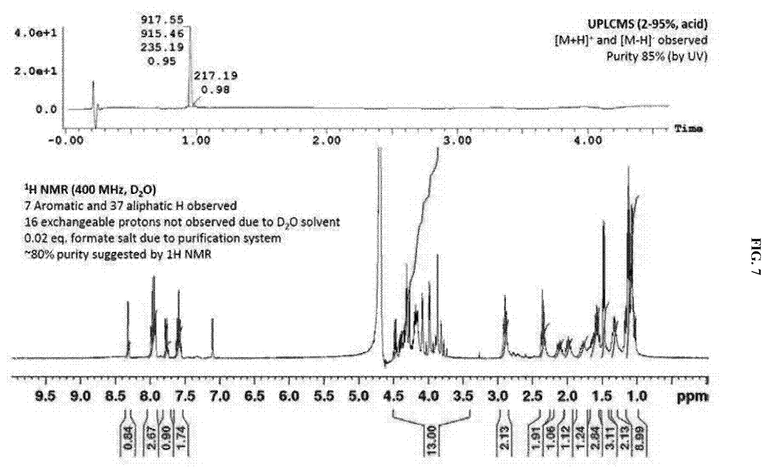

Seven types of AQTGTGKT analogs obtained by the above preparation methods were analyzed through ¹H NMR and ultraperformance liquid chromatography-mass spectrometry (UPLC-MS) techniques to confirm their chemical structures. The analysis results are shown in FIGS. 1 to 7, and all AQTGTGKT analogs including the compounds are summarized in Table 1.

TABLE 1

| Name | Chemical Formula |
|---|---|
| AQTGTGKT | 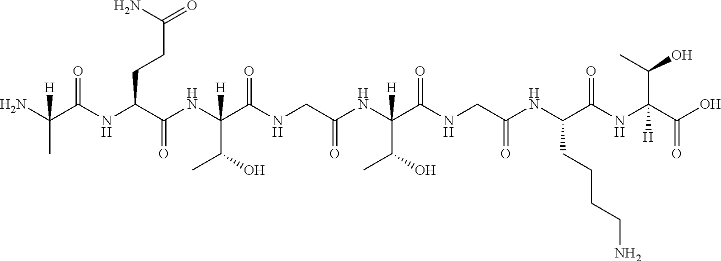 [SMILES: C[C@H](N)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 4-PhPh- AQTGTGKT | 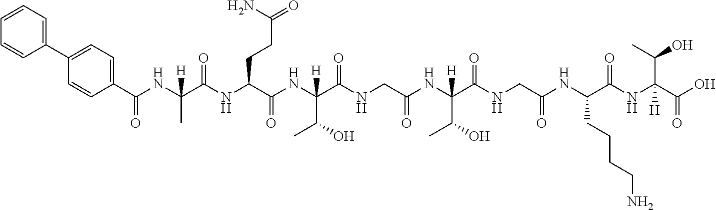 [SMILES: C[C@H](NC(=O)c1ccc(-c2ccccc2)cc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| Ac(Acyl)- AQTGTGKT | 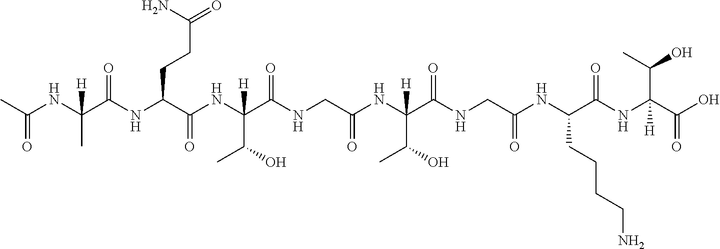 [SMILES: CC(=O)N[C@@H](C)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 3-PhPh- AQTGTGKT | 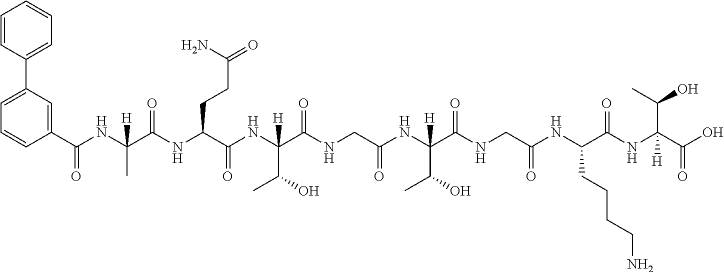 [SMILES: C[C@H](NC(=O)c1cccc(-c2ccccc2)c1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 4-MeOPh- AQTGTGKT | 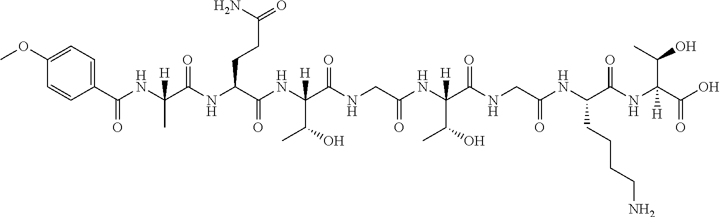 [SMILES: COc1ccc(C(=O)N[C@@H](C)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O)cc1] |
| 2-PhPh- AQTGTGKT | 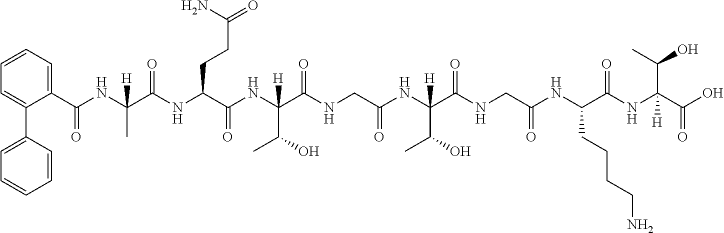 [SMILES: C[C@H](NC(=O)c1ccccc1-c1ccccc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| Ph- AQTGTGKT | 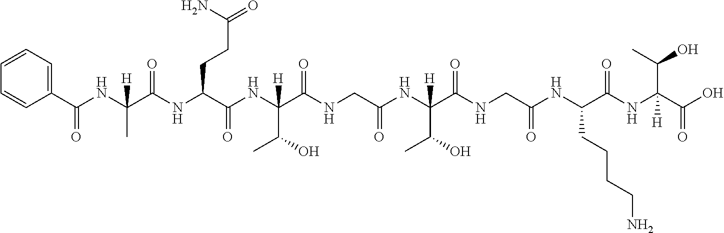 [SMILES: C[C@H](NC(=O)c1ccccc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| Naphthyl- AQTGTGKT | 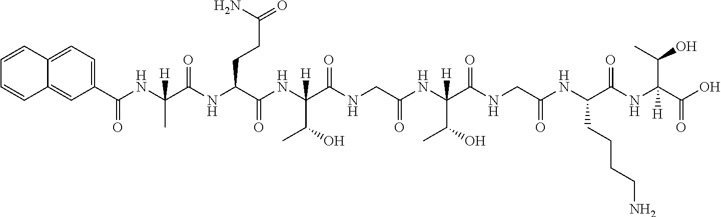 [SMILES: C[C@H](NC(=O)c1ccc2ccccc2c1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 2,4,6-MePh- AQTGTGKT | 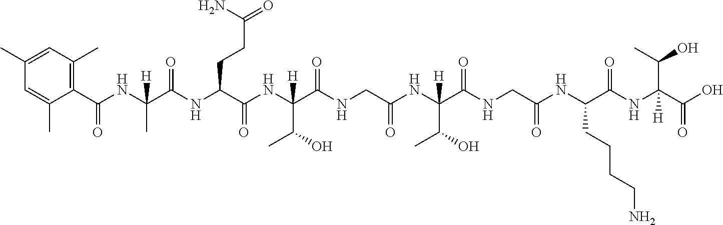 [SMILES: Cc1cc(C)c(C(=O)N[C@@H](C)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O)c(C)c1] |
| 1-MeOCF₃Ph- AQTGTGKT | 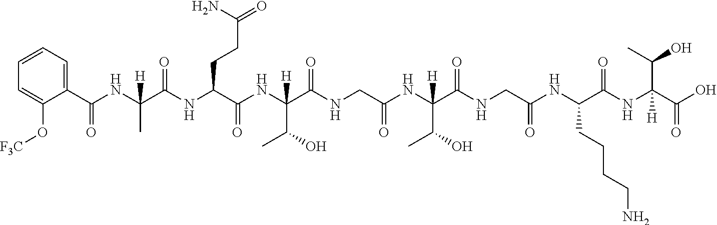 [SMILES: C[C@H](NC(=O)c1ccccc1OC(F)(F)F)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 4-MorPh- AQTGTGKT | 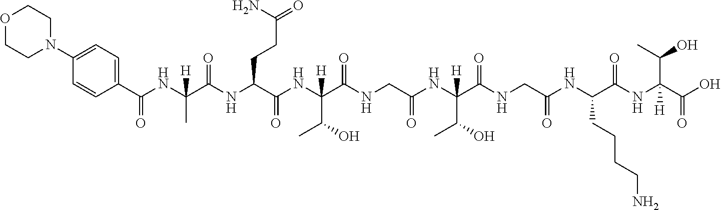 [SMILES: C[C@H](NC(=O)c1ccc(N2CCOCC2)cc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| PhMeMeC- AQTGTGKT | 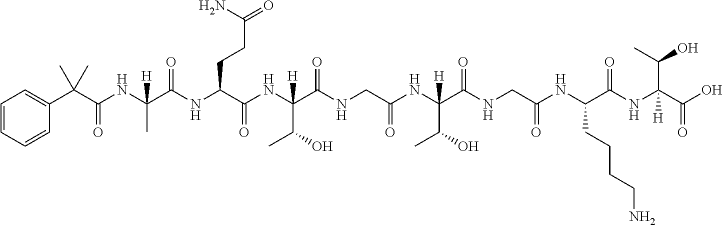 [SMILES: C[C@H](NC(=O)C(C)(C)c1ccccc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| 4-CF₃Ph- AQTGTGKT | 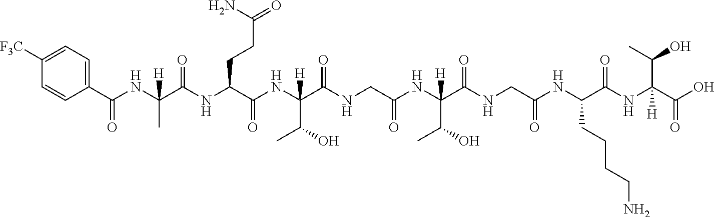 [SMILES: C[C@H](NC(=O)c1ccc(C(F)(F)F)cc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |
| Ph₃C- AQTGTGKT | 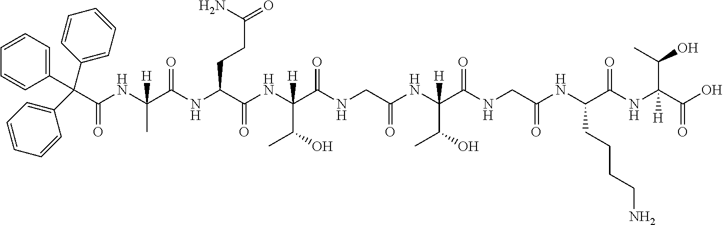 [SMILES: C[C@H](NC(=O)C(c1ccccc1)(c1ccccc1)c1ccccc1)C(=O)N[C@@H](CCC(N)=O)C(=O)N[C@H](C(=O)NCC(=O)N[C@H](C(=O)NCC(=O)N[C@@H](CCCCN)C(=O)N[C@H](C(=O)O)[C@@H](C)O)[C@@H](C)O)[C@@H](C)O] |

### Example 2. Confirmation of Effect of AQTGTGKT and Analog Thereof in Combination with Cancer Immunotherapy Drug

CT26 cancer cells in which CAGE expression was induced were mixed with Matrigel in a ratio of 1:1, and 200 μL of the cell mixture was injected into a female BALB/c mouse at a concentration of 1×10⁶ cells/mouse to produce a syngeneic mouse model. After cell inoculation was completed, when the volume of the formed tumor reached 70 to 120 mm³, groups were randomly separated and drug administration was performed. A cancer immunotherapy drug (anti-PD-1) was intraperitoneally administered at intervals of 3 days at a concentration of 10 mg/kg (mpk), and the compounds according to the present invention, AQTGTGKT, 3-PhPh-AQTGTGKT, and naphthyl-AQTGTGKT, were administered intravenously at intervals of 2 days at a concentration of 10 mpk. The size of the tumor was measured twice a week to calculate a tumor volume, and the weight of a subject was also measured. On day 15 after the start of administration, the tumor was removed, and the absolute weight was measured.

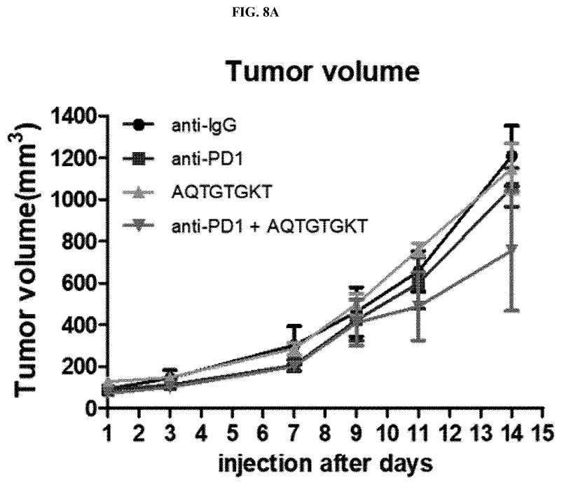

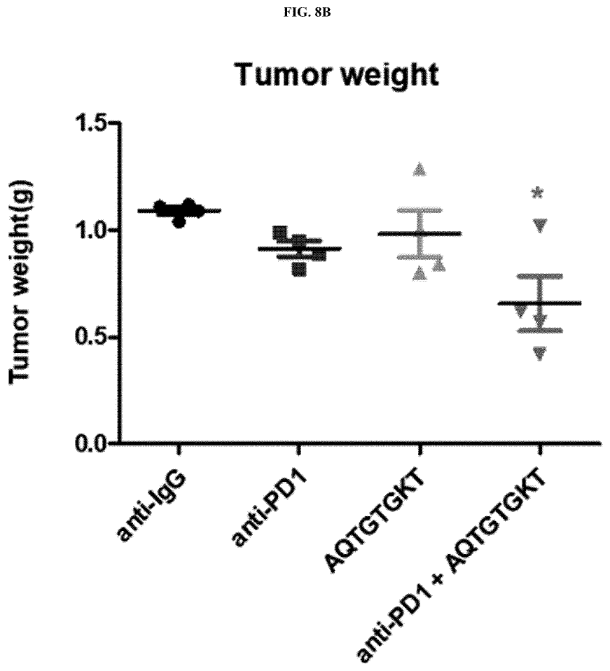

The results are shown in FIGS. 8A to 10B. First, the anticancer effects resulting from the single and combined administration of AQTGTGKT and the cancer immunotherapy drug were compared. There was no significant difference in tumor volume between the groups administered AQTGTGKT alone or anti-PD-1 alone and the control, indicating low tumor growth inhibition efficacy. On the other hand, in the group coadministered anti-PD-1 and AQTGTGKT, after day 11, tumor growth began to be suppressed compared to the control, and on day 15, it was confirmed that tumor growth was significantly suppressed in the coadministration group, compared to the control or single administration groups (FIG. 8A). As a result of removing the tumor from each subject and measuring the absolute weight of the tumor, although there was no significant difference between the tumor weight of the groups administered AQTGTGKT alone or anti-PD-1 alone and that of the control, the group coadministered anti-PD-1 and AQTGTGKT showed a significantly lower tumor weight than the control and the single administration groups (FIG. 8B). In addition, there were no subjects that lost weight or died during the experiment.

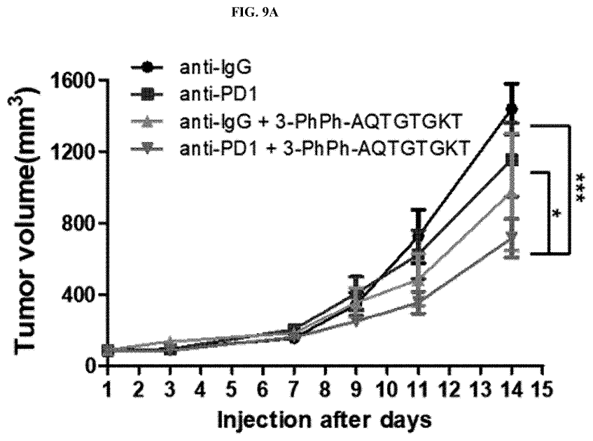

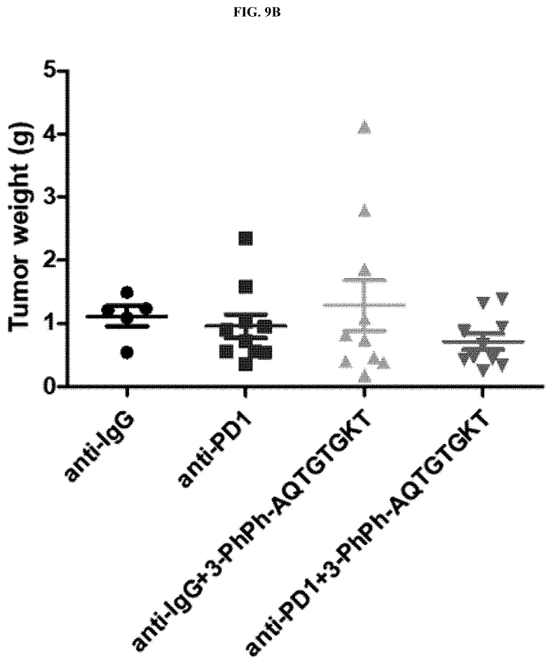

Next, the anticancer effects resulting from the single and coadministration of 3-PhPh-AQTGTGKT and the cancer immunotherapy drug were compared. There was no significant difference in tumor volume between the group administered anti-PD-1 alone and the control, indicating low tumor growth inhibition efficacy. On the other hand, in the group administered 3-PhPh-AQTGTGKT alone, after day 11, tumor growth began to decrease compared to the control, and particularly, in the group coadministered anti-PD-1 and 3-PhPh-AQTGTGKT, tumor growth was further suppressed, confirming that the efficacy of the coadministration was superior (FIG. 9A). In addition, as a result of removing the tumor from each subject and measuring the absolute weight of the tumor, although the tumor weight of the group administered anti-PD-1 alone was similar to that of the control, the tumor weight of the group administered 3-PhPh-AQTGTGKT alone was lower than that of the control, and the tumor weight of the group coadministered anti-PD-1 and 3-PhPh-AQTGTGKT was lower than those of the control or the single administration groups (FIG. 9B). Moreover, there were no subjects that lost weight or died during the experiment.

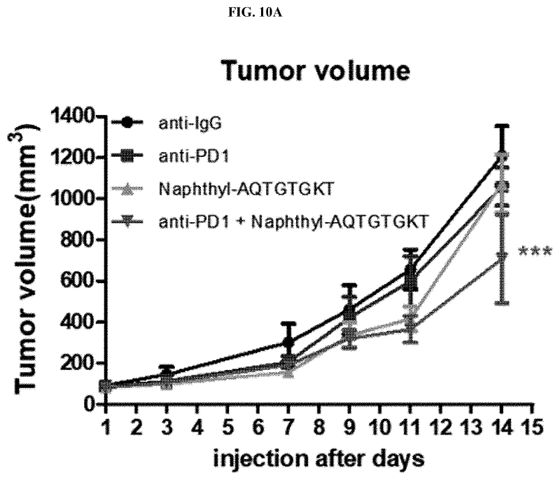

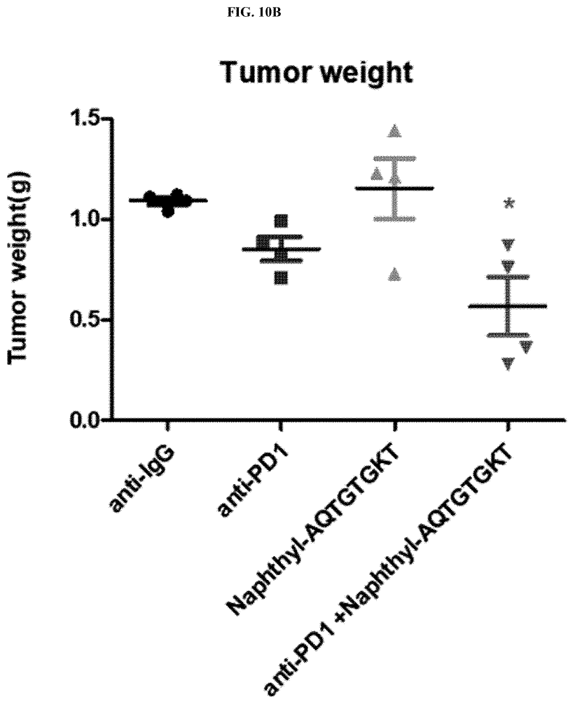

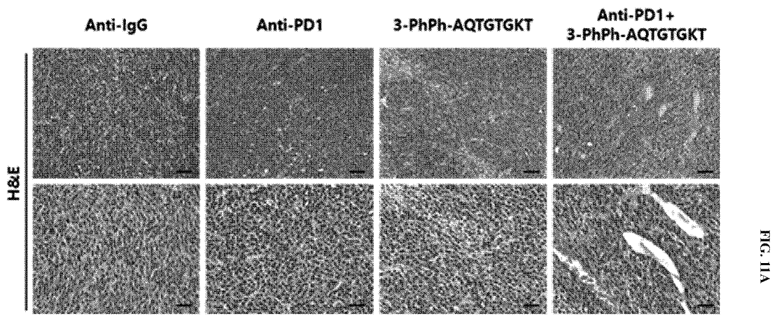

Subsequently, the anticancer effects resulting from the single and combined administration of Naphthyl-AQTGTGKT and the cancer immunotherapy drug were compared. There was no significant difference in tumor volume between the groups administered naphthyl-AQTGTGKT alone or anti-PD-1 alone and the control, indicating low tumor growth inhibition efficacy. On the other hand, in the group coadministered anti-PD-1 and naphthyl-AQTGTGKT, after day 11, tumor growth began to be suppressed compared to the control, and on day 15, it was confirmed that tumor growth was significantly suppressed in the coadministration group, compared to the control or single administration groups (FIG. 10A). As a result of removing the tumor from each subject and measuring the absolute weight of the tumor, although there was no significant difference between the tumor weight of the groups administered naphthyl-AQTGTGKT alone or anti-PD-1 alone and that of the control, the group coadministered anti-PD-1 and naphthyl-AQTGTGKT showed a significantly lower tumor weight than the control and the single administration groups (FIG. 10B). In addition, there were no subjects that lost weight or died during the experiment.

The above results show that, when the compounds according to the present invention are used in combination with a cancer immunotherapy drug, a more excellent anticancer effect is exhibited compared to monotherapy.

### Example 3. Confirmation of Anticancer Effect of AQTGTGKT and Analog Thereof Through Immune Cell Regulation

After administering the cancer immunotherapy drug and the compound of the present invention alone or in combination to a syngeneic mouse model produced using CT26 cells in which CAGE expression was induced, tumor tissue was isolated from each subject. The obtained tumor tissue was made into a paraffin block and then sectioned, and histological analysis was performed by performing H&E staining and microscopic observation on tissue slides. In addition, for immunochemical staining, primary antibodies specifically recognizing T cells or macrophages were treated, and secondary antibodies recognizing the primary antibodies and exhibiting fluorescence and then observed under a confocal microscope. Fluorescent images were taken at 20× magnification using a confocal microscope, and the number of T cells and macrophages (M2- and M1-type macrophages) on the entire screen was then measured and analyzed.

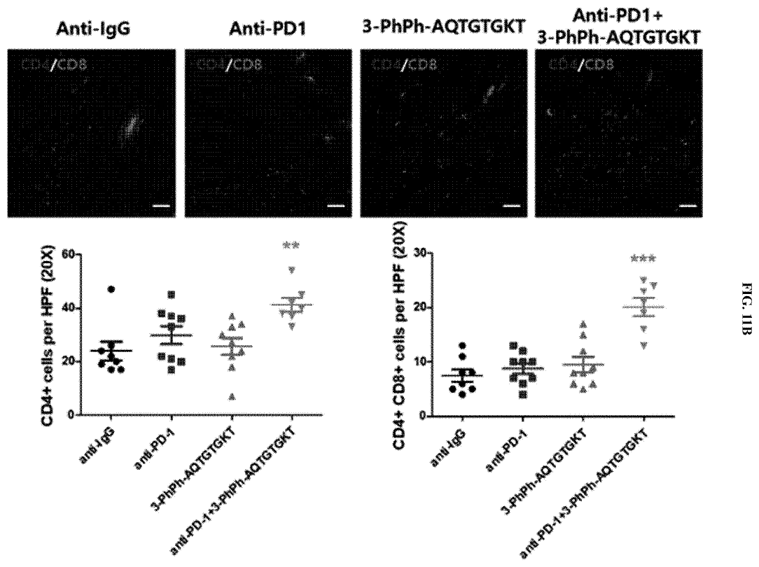

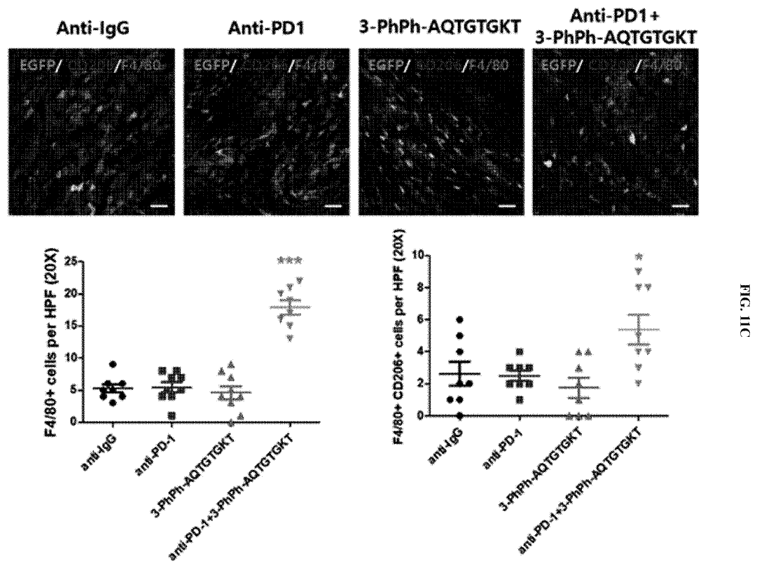

The results of histological analysis through H&E staining are shown in FIG. 11A. In the groups administered anti-IgG (Control) alone or anti-PD-1 (cancer immunotherapy drug) alone, tumor cells and cells constituting tumor tissue were observed to be very densely packed, and no particular death pattern of tumor cells was observed. In the group administered 3-PhPh-AQTGTGKT alone, some areas where tumor cells were killed were observed within the tumor tissue. In the tumor tissue of the group coadministered anti-PD-1 and 3-PhPh-AQTGTGKT, the tumor cells were actively killed, and the area showing cell death was observed to be quite large. In addition, in the area where cell death occurred, it was observed that many blood vessels had infiltrated and the size of the blood vessels was significantly expanded.

The results of immunochemical staining are shown in FIGS. 11B and 11C. As a result of analyzing the degree of tumor tissue infiltration of total T cells (CD4 T cells) and cytotoxic T cells (CD8 T cells), although no significant differences in the frequency of immune cells were observed between the tumor tissue of the groups administered anti-IgG (Control), anti-PD-1, or 3-PhPh-AQTGTGKT alone, in the tumor tissue of the group coadministered anti-PD-1 and 3-PhPh-AQTGTGKT, a statistically significant increase in frequency of total T cells and cytotoxic T cells was shown, compared to the control or the single administration groups (FIG. 11B). In addition, as a result of staining macrophages with antibodies specific for total macrophages (F4/80) or M2 macrophages (CD206) and then observing them, there was no significant difference between the frequencies of macrophages in the tumor tissue of the groups administered anti-PD-1 alone and 3-PhPh-AQTGTGKT alone, whereas statistically significant increases in both total macrophages and M2 macrophages were shown in the tumor tissue of the group coadministered anti-PD-1 and 3-PhPh-AQTGTGKT, compared to the control or single administration groups (FIG. 11C).

The above results show that the compound according to the present invention can promote the infiltration of immune cells, which suppress tumor growth, into tumor tissue, and particularly, when used in combination with a cancer immunotherapy drug, cancer cells can be effectively killed by further activating the function of the immune cells.

### Example 4. Confirmation of Effect of AQTGTGKT and Analog Thereof on Regulating Cytokine Production in Immune Cells

After administering the cancer immunotherapy drug and the compound of the present invention alone or in combination to a syngeneic mouse model produced using CT26 cells in which CAGE expression was induced, tumor tissue was isolated from each subject. RNA was extracted from the obtained tumor tissue, cDNA was synthesized, and then an mRNA expression level of the CXCL10 gene was measured through real-time PCR. For each mRNA, a relative expression level compared to the control was calculated through the 2^{-ΔΔ}^{^{Ct}} method.

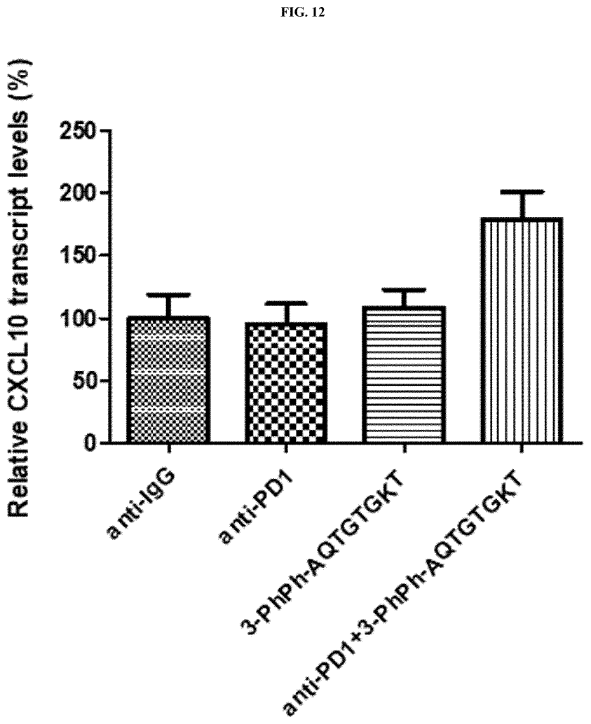

As a result, as shown in FIG. 12, CXCL10 mRNA expression levels were similar in the tumor tissue of the groups administered anti-IgG (Control), and anti-PD-1 alone or 3-PhPh-AQTGTGKT alone, whereas the tumor tissue of the group coadministered anti-PD-1 and 3-PhPh-AQTGTGKT showed a significantly increased mRNA expression level of CXCL10, compared to other groups (FIG. 12). The above results show that the coadministration of the compound of the present invention and the cancer immunotherapy drug exhibited an excellent immunoactivation effect compared to the single administration of each material.

### Example 5. Confirmation of Overall Survival (OS) Period According to Administration of AQTGTGKT and Analog Thereof

(1) Confirmation of Anticancer Effect Resulting from the Combined Use of 3-PhPh-AQTGTGKT and Anti-CTLA4

CT26 cancer cells in which CAGE expression was induced were mixed with Matrigel in a ratio of 1:1, and 200 μL of the cell mixture was injected into a female BALB/c mouse at a concentration of 1×10⁶ cells/mouse to produce a syngeneic mouse model. After cell inoculation was completed, when the volume of the formed tumor reached 70 to 120 mm³, groups were randomly separated and drug administration was performed. A cancer immunotherapy drug (anti-CTLA4) was intraperitoneally administered at intervals of 3 days at a concentration of 5 mpk, and the compound of the present invention (3-PhPh-AQTGTGKT) was intravenously administered at intervals of 2 days at a concentration of 10 mpk. The size of the tumor was measured twice a week to calculate a tumor volume, and the weight of a subject was also measured. On day 15 after the start of administration, the tumor was removed, and the absolute weight was measured.

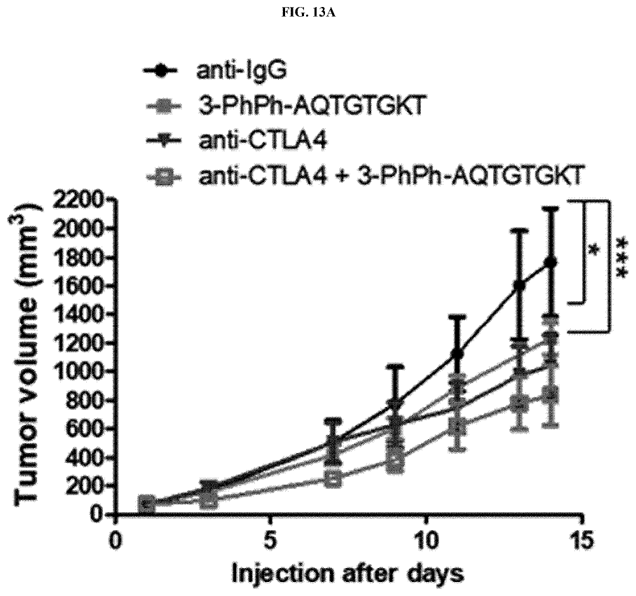

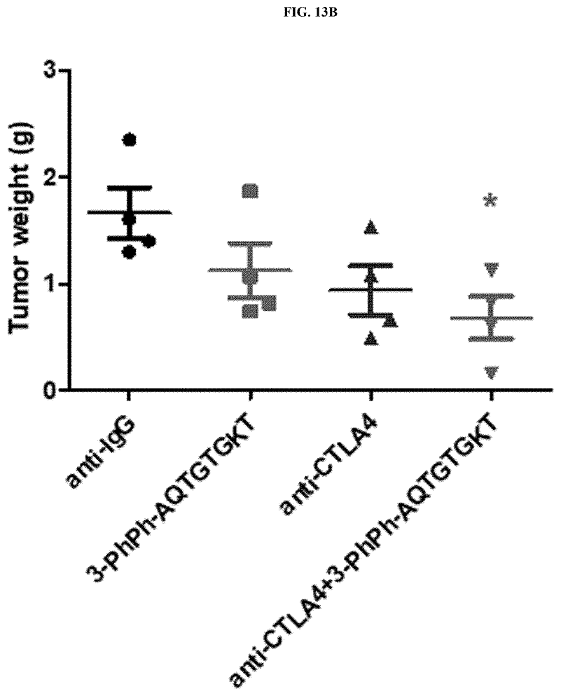

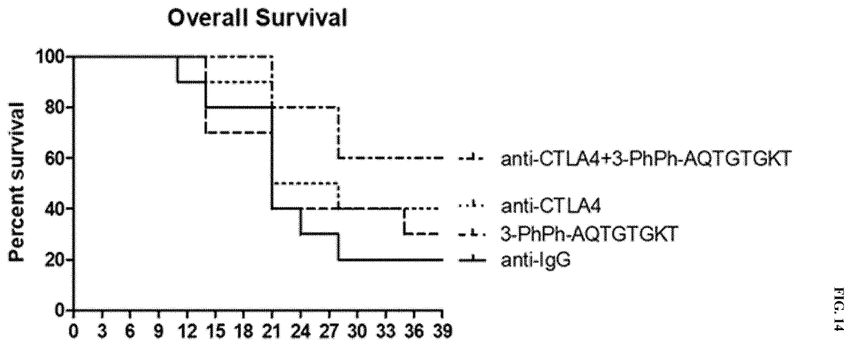

First, the anticancer effects resulting from the single and combined administration of the compound of the present invention and the cancer immunotherapy drug were compared. As a result, as shown in FIGS. 13A and 13B, from day 11 of administration, compared to the control, a statistically significant decrease in tumor growth was shown in the group administered anti-CTLA4 alone and the group administered 3-PhPh-AQTGTGKT alone, indicating that tumor growth was suppressed in the group administered anti-CTLA4 compared to the control. On the other hand, in the group coadministered anti-CTLA4 and 3-PhPh-AQTGTGKT, compared to the groups administered anti-CTLA4 alone or 3-PhPh-AQTGTGKT alone, tumor growth was further suppressed, confirming that the efficacy of the coadministration was superior to the single administration of each drug (FIG. 13A). In addition, as a result of removing the tumor from each subject and measuring the absolute weight of the tumor, the tumor weight of the group administered anti-CTLA4 alone was similar to that of the control, but the tumor weight of the group administered 3-PhPh-AQTGTGKT alone was lower than that of the control, confirming that tumor growth was more effectively suppressed. Further, it can be confirmed that the group coadministered anti-CTLA4 and 3-PhPh-AQTGTGKT showed a more significant reduction in tumor weight, compared to the groups administered each drug alone, confirming that the anticancer effect of coadministration is the best (FIG. 13B). In addition, there were no subjects that lost weight or died during the experiment.

### (2) Confirmation of Overall Survival Period According to Combined Use of 3-PhPh-AQTGTGKT and Anti-CTLA4

Next, an overall survival (OS) period according to the single or combined administration of the compound of the present invention and the cancer immunotherapy drug was compared. CT26 cancer cells in which CAGE expression was induced were mixed with Matrigel in a ratio of 1:1, and 200 μL of the cell mixture was injected into a female BALB/c mouse at a concentration of 1×10⁶ cells/mouse to produce a syngeneic mouse model. After cell inoculation was completed, when the volume of the formed tumor reached 70 to 120 mm³, groups were randomly separated and drug administration was performed. The cancer immunotherapy drug (anti-CTLA4) was administered at intervals of 3 days at a concentration of 5 mpk, and the compound of the present invention (3-PhPh-AQTGTGKT) was intravenously administered at intervals of 2 days at a concentration of 10 mpk, and the survival rate of the subjects was analyzed using Kaplan Meier analysis.

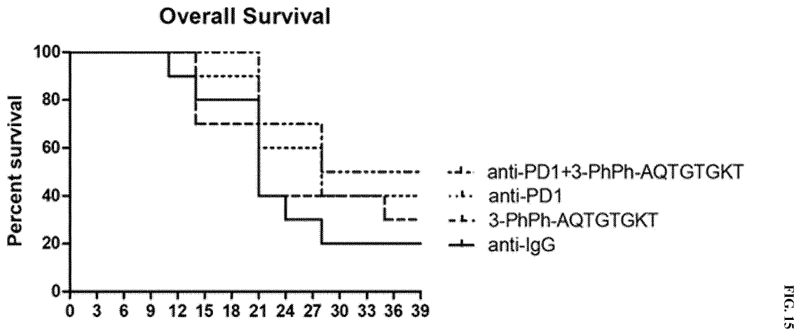

As a result of analyzing the survival curves, for 39 days, in the control, 8 out of 10 subjects died and two survived, and in the group administered anti-CTLA4 alone, 6 out of 10 subjects died and 4 survived. In addition, in the group administered 3-PhPh-AQTGTGKT alone, 7 out of 10 subjects died and 3 survived. On the other hand, in the group coadministered anti-CTLA4 and 3-PhPh-AQTGTGKT, only 4 out of 10 subjects died and 6 survived, indicating that the number of surviving subjects increased compared to the control and the single administration groups (FIG. 14). The above results show that the coadministration of the compound of the present invention and the cancer immunotherapy drug can further increase the survival rate of subjects compared to the single administration of each material.

### (3) Confirmation of Overall Survival Period According to Combined Use of 3-PhPh-AQTGTGKT and Anti-PD-1

Subsequently, the effect of the coadministration of the compound of the present invention and a different cancer immunotherapy drug on the survival rate of subjects was confirmed. CT26 cancer cells in which CAGE expression was induced were mixed with Matrigel in a ratio of 1:1, and 200 μL of the cell mixture was injected into a female BALB/c mouse at a concentration of 1×10⁶ cells/mouse to produce a syngeneic mouse model. After cell inoculation was completed, when the volume of the formed tumor reached 70 to 120 mm³, groups were randomly separated and drug administration was performed. The cancer immunotherapy drug (anti-PD-1) was administered at intervals of 3 days at a concentration of 10 mpk, and the compound of the present invention (3-PhPh-AQTGTGKT) was intravenously administered at intervals of 2 days at a concentration of 10 mpk, and the survival rate of the subjects was analyzed using Kaplan Meier analysis.

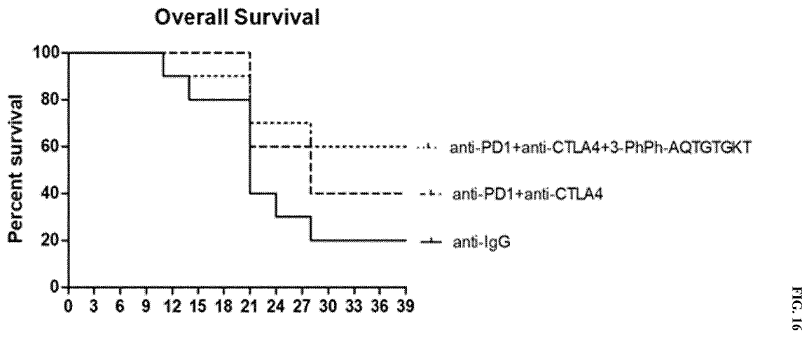

As a result, as shown in FIG. 15, for 39 days, in the control, 8 out of 10 subjects died and two survived, and in the group administered anti-PD-1 alone, 6 out of 10 subjects died and 4 survived. In addition, in the group administered 3-PhPh-AQTGTGKT alone, 7 out of 10 subjects died and 3 survived. On the other hand, in the group coadministered anti-PD-1 and 3-PhPh-AQTGTGKT, 5 out of 10 subjects died and 5 survived. Therefore, it was confirmed that the group coadministered anti-PD1 and 3-PhPh-AQTGTGKT shows a higher survival rate of subjects than the control and the single administration groups (FIG. 15). The above results prove again that the coadministration of the compound of the present invention and the cancer immunotherapy drug is more effective in increasing the survival rate of subjects, compared to the single administration of each material.

### (4) Confirmation of Overall Survival Period According to Combined Use of 3-PhPh-AQTGTGKT, Anti-CTLA4, and Anti-PD-1

CT26 cancer cells in which CAGE expression was induced were mixed with Matrigel in a ratio of 1:1, and 200 μL of the cell mixture was injected into a female BALB/c mouse at a concentration of 1×10⁶ cells/mouse to produce a syngeneic mouse model. After cell inoculation was completed, when the volume of the formed tumor reached 70 to 120 mm³, groups were randomly separated and drug administration was performed. The groups were divided into a control; a group coadministered anti-PD-1+anti CTLA4; and group coadministered anti-PD-1+anti-CTLA4+3-PhPh-AQTGTGKT. Anti-PD-1 and anti-CTLA4 were intraperitoneally administered at intervals of 3 days at concentrations of 10 mpk and 5 mpk, respectively, and 3-PhPh-AQTGTGKT was intravenously administered at intervals of 2 day at a concentration of 10 mpk, and then the survival rate of the subjects was analyzed using Kaplan Meier analysis.

As a result, as shown in FIG. 16, for 39 days, in the control, 8 out of 10 subjects died and 2 survived, and in the group coadministered anti-PD1 and anti-CTLA4, 6 out of 10 subjects died and 4 survived. On the other hand, in the group coadministered anti-PD-1, anti-CTLA4, and 3-PhPh-AQTGTGKT, 4 out of 10 subjects died and 6 survived (FIG. 16). That is, the above results show that, when the compound according to the present invention was coadministered with two or more types of cancer immunotherapy drugs, the survival rate of subjects can be further increased.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive.

### INDUSTRIAL APPLICABILITY

The present invention relates to a pharmaceutical composition for immune enhancement, and has been completed by confirming that the oligopeptide AQTGTGKT and an analog thereof have an effect of appropriately regulating immune activity in the body, for example, enhancing immune activity for body defense and suppressing excessive immune responses in the case of cancer or an infectious disease. Particularly, the compound according to the present invention can significantly enhance the sensitivity of a subject to a cancer immunotherapy drug, and adjust a physiological balance by activating immune cells in a tumor microenvironment through the regulation of cytokines or chemokines and suppressing excessive autophagy activity, thereby maximizing its anticancer effect in combination with the cancer immunotherapy drug. Therefore, since the compound according to the present invention can optimize the defense function of the body and enhance the effect of the cancer immunotherapy drug through the regulation of immune responses, it is expected to be used in the treatment of various immune diseases and cancer.

1. A method for immune enhancement, comprising: administering to a subject in need thereof a pharmaceutically effective amount of an oligopeptide consisting of the amino acid sequence of SEQ ID NO: 1 or an analog thereof.

2. The method of claim 1, wherein the analog is a compound represented by General Formula X-AQTGTGKT, in which X is a compound represented by Chemical Formula 1 below, or a pharmaceutically acceptable salt thereof: 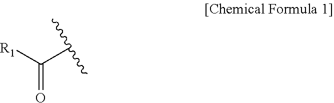
in Chemical Formula 1,
R₁ is hydrogen, a substituted or unsubstituted phenyl group, a substituted or unsubstituted naphthyl group, or a substituted or unsubstituted C₁ to C₅ alkyl group; or wherein R₁ is a compound represented by any one of Chemical Formulas 2 to 4 below: 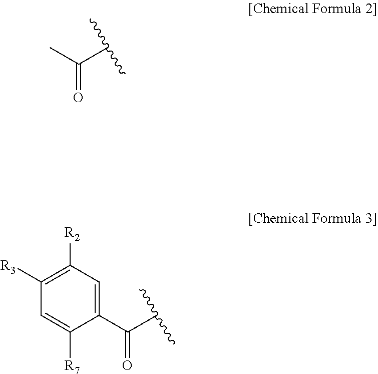
(in Chemical Formula 3,
R₂ and R₄ are each independently hydrogen, a C₁ to C₅ alkyl group, or a phenyl group; and
R₃ is hydrogen, a C₁ to C₅ alkyl group, a phenyl group, or a C₁ to C₃ alkoxy group) 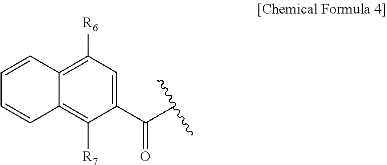
(in Chemical Formula 4,
R₆ and R₇ are each independently hydrogen or a C₁ to C₃ alkyl group).

3. (canceled)

4. The method of claim 1, wherein the oligopeptide or an analog thereof satisfies one or more characteristics selected from the group consisting of the following:
(a) increasing an immune response against one or more selected from the group consisting of tumor antigens, bacterial infections, and viral infections;
(b) suppressing immune hypersensitivity;
(c) regulating the level or activity of one or more selected from the group consisting of CXCL10, IFNγ, TNFα, CCL5, CXCL9, CXCL11, IL-1α, IL-1β, IL-6, and IL-12; and
(d) increasing the level or activity of one or more selected from the group consisting of cytotoxic T cells and M2 macrophages.

5. A method of treating cancer, comprising: administering to a subject in need thereof a pharmaceutically effective amount of a composition comprising an oligopeptide consisting of the amino acid sequence of SEQ ID NO: 1 or an analog thereof, and a cancer immunotherapy drug as active ingredients.

6. The method of claim 5, wherein the analog is a compound represented by the general formula X-AQTGTGKT, in which X is a compound represented by Chemical Formula 1 below, or a pharmaceutically acceptable salt thereof: 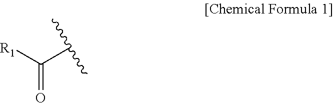
in Chemical Formula 1,
R₁ is hydrogen, a substituted or unsubstituted phenyl group, a substituted or unsubstituted naphthyl group, or a substituted or unsubstituted C₁ to C₅ alkyl group; or wherein R₁ is a compound represented by any one of Chemical Formulas 2 to 4 below: 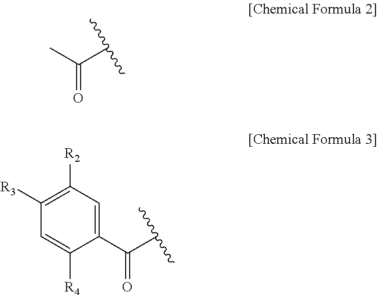
(in Chemical Formula 3,
R₂ and R₄ are each independently hydrogen, a C₁ to C₅ alkyl group, or a phenyl group; and
R₃ is hydrogen, a C₁ to C₅ alkyl group, a phenyl group, or a C₁ to C₃ alkoxy group) 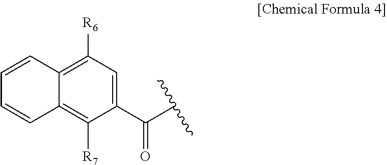
(in Chemical Formula 4,
R₆ and R₇ are each independently hydrogen or a C₁ to C₃ alkyl group).

7. (canceled)

8. The method of claim 5, wherein the cancer immunotherapy drug is one or more selected from the group consisting of an immune checkpoint inhibitor, a costimulatory molecule agonist, a cytokine therapeutic, a CAR-T cell therapeutic, and an autologous CD8⁺ T immune cell therapeutic.

9. The method of claim 8, wherein the immune checkpoint inhibitor is one or more selected from the group consisting of a PD-L1 inhibitor, a PD-1 inhibitor, a CTLA-4 inhibitor, a LAG3 inhibitor, a TIM3 inhibitor, a 4-1BB inhibitor, a LAG-3 inhibitor, a B7-H4 inhibitor, an HVEM inhibitor, a TIM4 inhibitor, a GAL9 inhibitor, a VISTA inhibitor, a KIR inhibitor, a TIGIT inhibitor, and a BTLA inhibitor.

10. The method of claim 8, wherein the immune checkpoint inhibitor is one or more selected from the group consisting of atezolizumab, avelumab, dostarlimab, durvalumab, ipilimumab, nivolumab, and pembrolizumab.

11. The method of claim 8, wherein the costimulatory molecule agonist is one or more selected from the group consisting of a 4-1BB inhibitor, and an OX40 inhibitor.

12. The method of claim 5, wherein the composition is in the form of a mixture in which the oligopeptide or an analog thereof, and the cancer immunotherapy drug are mixed.

13. The method of claim 5, wherein the composition is designed such that each of the oligopeptide or analog thereof and the cancer immunotherapy drug is prepared and simultaneously, separately, or sequentially administered.

14. The method of claim 5, wherein the composition is administered to a subject at a dose such that the cancer immunotherapy drug is administered at a concentration of 0.1 to 50 mg/kg.

15. The method of claim 5, wherein the composition is administered to a subject at a dose such that the oligopeptide or an analog thereof is administered at a concentration of 0.01 to 500 mg/kg.

16. The method of claim 5, wherein the weight ratio of the cancer immunotherapy drug:the oligopeptide or an analog thereof is 1:0.1 to 10.

17. The method of claim 5, wherein the composition satisfies one or more selected from the group consisting of the following:
(a) an increase in the level or activity of tumor-suppressive immune cells;
(b) a reduction in the level of immunosuppressive immune cells; and
(c) the regulation of the level or activity of one or more selected from the group consisting of CXCL10, IFNγ, TNFα, CCL5, CXCL9, CXCL11, IL-1α, IL-1β, IL-6, and IL-12.

18. The method of claim 5, wherein the cancer is one or more selected from the group consisting of squamous cell carcinoma, lung cancer, adenocarcinoma of the lung, peritoneal cancer, skin cancer, skin or intraocular melanoma, rectal cancer, perianal cancer, esophageal cancer, small intestine cancer, endocrine cancer, parathyroid cancer, adrenal cancer, soft tissue sarcoma, urethral cancer, blood cancer, liver cancer, gastrointestinal cancer, pancreatic cancer, glioblastoma, cervical cancer, ovarian cancer, bladder cancer, hepatocellular carcinoma, breast cancer, colon cancer, colorectal cancer, endometrial cancer, uterine cancer, salivary gland cancer, kidney cancer, prostate cancer, vulvar cancer, thyroid cancer, head and neck cancer, and brain cancer; or
wherein the cancer is one or more selected from the group consisting of microsatellite instability-high (MSI-H) mutation, microsatellite instability-low (MSI-L) mutation, and microsatellite stability (MSS) mutation.

19. (canceled)

20. The method of claim 5, wherein the composition includes two or more types of cancer immunotherapy drugs.

21. (canceled)

22. A method for enhancing the anticancer effect of a cancer immunotherapeutic agent, comprising: administering to a subject in need thereof a pharmaceutically effective amount of an oligopeptide consisting of the amino acid sequence of SEQ ID NO: 1 or an analog thereof.

23. The method of claim 22, wherein the analog is a compound represented by the general formula X-AQTGTGKT, in which X is a compound represented by Chemical Formula 1 below, or a pharmaceutically acceptable salt thereof: 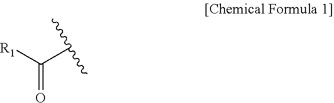
in Chemical Formula 1,
R₁ is hydrogen, a substituted or unsubstituted phenyl group, a substituted or unsubstituted naphthyl group, or a substituted or unsubstituted C₁ to C₅ alkyl group; or wherein R₁ is represented by any one of Chemical Formulas 2 to 4 below: 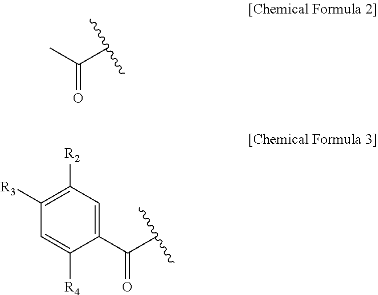
(in Chemical Formula 3,
R₂ and R₄ are each independently hydrogen, a C₁ to C₅ alkyl group, or a phenyl group; and
R₃ is hydrogen, a C₁ to C₅ alkyl group, a phenyl group, or a C₁ to C₃ alkoxy group) 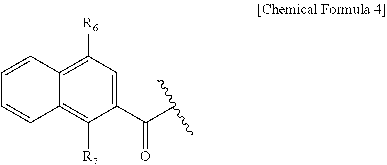
(in Chemical Formula 4,
R₆ and R₇ are each independently hydrogen, or a C₁ to C₃ alkyl group).

24. (canceled)

25. The method of claim 22, wherein the composition is simultaneously, separately, or sequentially administered with a cancer immunotherapy drug.

26-33. (canceled)
